(19) United States
(12) Patent Application Publication
(10) Pub. No.: US 2010/0292224 A1
(43) Pub. Date: Nov. 18, 2010

(54) ISOTHIAZOLIDINE 1,1-DIOXIDE AND TETRAHYDRO-2H-1,2-THIAZINE 1,1-DIOXIDE DERIVATIVES AS P2X7 MODULATORS

(75) Inventor: Daryl Simon Walter, Essex (GB)

Correspondence Address: GlaxoSmithKline;GLOBAL PATENTS -US, UW2220, P. O. BOX 1539, KING OF PRUSSIA, PA, 19406-0939, US

(73) Assignee: GLAXO GROUP LIMITED, Greenford Middlesex (GB)

(21) Appl. No.: 12/747,995

(22) PCT Filed: Dec. 5, 2008
(86) PCT No.: PCT/EP2008/066945

§ 371 Date: Jun. 14, 2010

(30) Foreign Application Priority Data
Dec. 18, 2007 (GB) 0724625.9

(51) Int. Cl. A61K 31/54; C07D 275/03; A61K 31/425; C07D 279/02; A61P 29/00; A61P 25/28; A61P 25/00; A61P 25/14

(52) U.S. Cl. 514/222.2; 548/214; 514/372; 544/3

(57) ABSTRACT

The present invention provides a compound of formula (1) or a pharmaceutically acceptable salt thereof:
wherein R¹ represents optionally substituted C₁₋₄ alkyl; X represents —(CR¹⁰R¹¹)ₙ— in which n represents 1 or 2; R², R³ and R⁴ independently represent H, F or Me; and R⁵; R⁶; R⁷; R⁸ and R⁹ independently represent hydrogen, halogen, or cyano; or optionally substituted C₁₋₆ alkyl, C₂₋₆ alkenyl, C₂₋₆ alkynyl or C₃₋₆ cycloalkyl, such that at least one of R⁵ and R⁹ represents a group other than hydrogen. The compounds or salts are thought to modulate P2X7 receptor function and to be capable of antagonizing the effects of ATP at the P2X7 receptor. The invention also provides the use of the compound or salt in the treatment or prevention of inflammatory pain, neuropathic pain, visceral pain, rheumatoid arthritis or osteoarthritis.

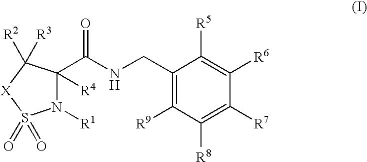

The present invention relates to isothiazolidine 1,1-dioxide derivatives or tetrahydro-2H-1,2-thiazine 1,1-dioxide derivatives which modulate P2X7 receptor function and are capable of antagonizing the effects of ATP at the P2X7 receptor (“P2X7 receptor antagonists”); to processes for their preparation; to pharmaceutical compositions containing them; and to the use of such compounds in therapy.

The P2X7 receptor is a ligand-gated ion-channel which is expressed in cells of the hematopoietic lineage, e.g. macrophages, microglia, mast cells, and lymphocytes (T and B) (see, for example, Collo, et al. Neuropharmacology, Vol. 36, pp 1277-1283 (1997)), and is activated by extracellular nucleotides, particularly adenosine triphosphate (ATP). Activation of P2X7 receptors has been implicated in giant cell formation, degranulation, cytolytic cell death, CD62L shedding, regulation of cell proliferation, and release of proinflammatory cytokines such as interleukin 1 beta (IL-1β) (e.g. Ferrari, et al., J. Immunol., Vol. 176, pp 3877-3883 (2006)), interleukin 18 (IL-18), and tumour necrosis factor alpha (TNFα) (e.g. Hide, et al. Journal of Neurochemistry, Vol. 75, pp 965-972 (2000)). P2X7 receptors are also located on antigen presenting cells, keratinocytes, parotid cells, hepatocytes, erythrocytes, erythroleukaemic cells, monocytes, fibroblasts, bone marrow cells, neurones, and renal mesangial cells. Furthermore, the P2X7 receptor is expressed by presynaptic terminals in the central and peripheral nervous systems and has been shown to mediate glutamate release in glial cells (Anderson, C. et al. Drug. Dev. Res., Vol. 50, page 92 (2000)).

The localisation of the P2X7 receptor to key cells of the immune system, coupled with its ability to release important inflammatory mediators from these cells suggests a potential role of P2X7 receptor antagonists in the treatment of a wide range of diseases including pain and neurodegenerative disorders. Recent preclinical in vivo studies have directly implicated the P2X7 receptor in both inflammatory and neuropathic pain (Dell'Antonio et al., Neurosci. Lett., Vol. 327, pp 87-90 (2002),. Chessell, I P., et al., Pain, Vol. 114, pp 386-396 (2005), Honore et al., J. Pharmacol. Exp. Ther., Vol. 319, p 1376-1385 (2006)) while there is in vitro evidence that P2X7 receptors mediate microglial cell induced death of cortical neurons (Skaper, S. D., et al., Glia, Vol. 54, p 234-242 (2006)). In addition, up-regulation of the P2X7 receptor has been observed around β-amyloid plaques in a transgenic mouse model of Alzheimer's disease (Parvathenani, L. et al. J. Biol. Chem., Vol. 278 (15), pp 13309-13317 (2003)).

WO 98/13355 (Guilford Pharma Inc) describe a series of heterocyclic ester or amide compounds which are claimed to be useful as neurotrophic compounds having an affinity for FKBP-type immunophilins, and their use as inhibitors of, for example, peptidyl-prolyl isomerase or rotamase enzyme activity.

The present invention provides compounds which modulate P2X7 receptor function and are capable of antagonizing the effects of ATP at the P2X7 receptor (“P2X7 receptor antagonists”).

In a first aspect of the invention, there is provided a compound of formula (1) or a pharmaceutically acceptable salt thereof:

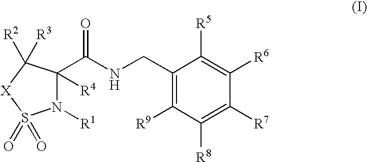

wherein:
R¹ represents C₁₋₄ alkyl optionally substituted with 1, 2 or 3 halogen (e.g. fluorine) atoms;
X represents —(CR¹⁰R¹¹)ₙ—;
R¹⁰ and R¹¹ independently represent hydrogen, halogen, or C₁₋₆ alkyl; wherein said C₁₋₆ alkyl group may be optionally substituted with 1, 2 or 3 halogen (e.g. fluorine) atoms;
n represents an integer being 1 or 2, such that when n represents 2, R¹ represents a group other than isobutyl;
R², R³ and R⁴ independently represent hydrogen, fluorine or methyl; and
R⁵, R⁶, R⁷, R⁸ and R⁹ independently represent hydrogen, halogen, cyano, C₁₋₆ alkyl, C₂₋₆ alkenyl, C₂₋₆ alkynyl, C₃₋₆ cycloalkyl, wherein any of said C₁₋₆ alkyl, C_{2-6 alkenyl, C}₂₋₆ alkynyl or C₃₋₆ cycloalkyl groups may be optionally substituted with 1, 2 or 3 halogen (e.g. fluorine) atoms, such that at least one of R⁵ and R⁹ represents a group other than hydrogen.

As used herein, the term “alkyl” (when used as a group or as part of a group) refers to a straight or branched hydrocarbon chain containing the specified number of carbon atoms. For example, C₁₋₆ alkyl means a straight or branched hydrocarbon chain containing at least 1 and at most 6 carbon atoms. Examples of alkyl and C₁₋₆ alkyl include, but are not limited to: methyl (Me), ethyl (Et), n-propyl, isopropyl, n-butyl, isobutyl, sec-butyl, t-butyl, n-hexyl and isohexyl.

“C₃₋₆cycloalkyl” groups can be cyclopropyl, cyclobutyl, cyclopentyl, or cyclohexyl.

The term ‘halogen’ is used herein to mean, unless otherwise stated, a group which is fluorine, chlorine, bromine or iodine.

It is to be understood that the present invention covers and discloses all possible combinations of particular, preferred, suitable, or other embodiments of groups or features (e.g. of R¹, R², R³, R⁴, R⁵, R⁶, R⁷, R⁸, R⁹, R¹⁰, R¹¹, X, and/or n), e.g. covers and discloses all possible combinations of embodiments of different groups or features, which embodiments are described herein.

In certain particular embodiments, X represents —CH₂— or —(CH₂)₂—. Preferably, X represents —CH₂—.

Preferably, n represents 1.

In certain particular embodiments, R¹ represents C₁₋₄ alkyl (e.g. methyl, ethyl, isopropyl or isobutyl).

Preferably, R¹ represents C₁₋₃ alkyl, in particular methyl, ethyl or isopropyl. More preferably, R¹ represents methyl or ethyl.

When n represents 2, in certain particular embodiments, R¹ is C₁₋₃ alkyl (e.g. methyl, ethyl or isopropyl), more particularly methyl or ethyl, still more particularly methyl.

In certain particular embodiments, R², R³ and R⁴ each represent hydrogen.

In certain particular embodiments, R⁵ represents hydrogen, halogen (e.g. fluorine or chlorine) or C₁₋₆ alkyl (e.g. methyl) optionally substituted with 1, 2 or 3 halogen atoms (e.g. —CF₃).

In certain particular embodiments, R⁶ represents hydrogen or halogen (e.g. fluorine or chlorine).

In certain particular embodiments, R⁷ represents hydrogen or halogen (e.g. fluorine or chlorine).

In certain particular embodiments, R⁸ represents hydrogen, halogen (e.g. fluorine or chlorine) or C₁₋₆ alkyl optionally substituted with 1, 2 or 3 halogen atoms (e.g. —CF₃).

In certain particular embodiments, R⁹ represents hydrogen, halogen (e.g. fluorine or chlorine) or C₁₋₆ alkyl (e.g. methyl) optionally substituted with 1, 2 or 3 halogen atoms (e.g. —CF₃).

### Preferably,

R⁵ represents hydrogen, fluorine, chlorine, methyl, or —CF₃;
R⁶ represents hydrogen, fluorine or chlorine;
R⁷ represents hydrogen, fluorine or chlorine;
R⁸ represents hydrogen, fluorine, chlorine, or —CF₃; and
R⁹ represents hydrogen, fluorine, chlorine, methyl or —CF₃, such that at least one of R⁵ and R⁹ represents a group other than hydrogen.

Preferably, R⁵, R⁶, R⁷, R⁸ and R⁹ are as defined in one or more of the Examples disclosed herein.

More preferably,
R⁵ represents chlorine; R⁶ and R⁸ and R⁹ represent hydrogen; and R⁷ represents chlorine; or
R⁵ represents chlorine; R⁶ and R⁸ and R⁹ represent hydrogen; and R⁷ represents fluorine; or
R⁵ represents chlorine; and R⁶ and R⁷ represents fluorine; and R⁸ and R⁹ represent hydrogen; or
R⁵, R⁶ and R⁷ represent hydrogen; R⁸ represents —CF₃; and R⁹ represents chlorine.

In one particular embodiment of the invention, there is provided a compound of formula (1), or a pharmaceutically acceptable salt thereof, which is:
N-{[4-fluoro-2-(trifluoromethyl)phenyl]methyl}-2-(2-methylpropyl)-3-isothiazolidinecarboxamide 1,1-dioxide (E1);
N-[(2-chloro-4-fluorophenyl)methyl]-2-(2-methylpropyl)-3-isothiazolidinecarboxamide 1,1-dioxide (E2);
N-[(2-chloro-4-fluorophenyl)methyl]-2-methyl-3-isothiazolidinecarboxamide 1,1-dioxide (E3);
N-[(2,4-dichlorophenyl)methyl]-2-(2-methylpropyl)-3-isothiazolidinecarboxamide 1,1-dioxide (E4);
N-[(2-chloro-6-methylphenyl)methyl]-2-(2-methylpropyl)-3-isothiazolidinecarboxamide 1,1-dioxide (E5);
N-[(2-chlorophenyl)methyl]-2-(1-methylethyl)-3-isothiazolidinecarboxamide 1,1-dioxide (E6);
N-[(2-chlorophenyl)methyl]-2-ethyl-3-isothiazolidinecarboxamide 1,1-dioxide (E7); N-{[4-fluoro-2-(trifluoromethyl)phenyl]methyl}-2-(1-methylethyl)-3-isothiazolidinecarboxamide 1,1-dioxide (E8);
N-[(2-chlorophenyl)methyl]-2-methyl-3-isothiazolidinecarboxamide 1,1-dioxide (E9);
N-{[4-fluoro-2-(trifluoromethyl)phenyl]methyl}-2-methyl-3-isothiazolidinecarboxamide 1,1-dioxide (E10);
N-[(2-chlorophenyl)methyl]-2-(2-methylpropyl)-3-isothiazolidinecarboxamide 1,1-dioxide (E11);
N-[(2-chloro-6-methylphenyl)methyl]-2-methyl-3-isothiazolidinecarboxamide 1,1-dioxide (E12);
N-[(2,4-dichlorophenyl)methyl]-2-methyl-3-isothiazolidinecarboxamide 1,1-dioxide (E13);
2-ethyl-N-[(2,3,4-trifluorophenyl)methyl]-3-isothiazolidinecarboxamide 1,1-dioxide (E14);
N-[(2-chloro-4-fluorophenyl)methyl]-2-ethyl-3-isothiazolidinecarboxamide 1,1-dioxide (E15);
2-methyl-N-[(2,3,4-trifluorophenyl)methyl]-3-isothiazolidinecarboxamide 1,1-dioxide (E16);
N-[(3-chloro-2-methylphenyl)methyl]-2-methyl-3-isothiazolidinecarboxamide 1,1-dioxide (E17);
N-[(3-chloro-2-methylphenyl)methyl]-2-(1-methylethyl)-3-isothiazolidinecarboxamide 1,1-dioxide (E18);
N-[(3-chloro-2-methylphenyl)methyl]-2-(2-methylpropyl)-3-isothiazolidinecarboxamide 1,1-dioxide (E19);
N-[(3-chloro-2-methylphenyl)methyl]-2-ethyl-3-isothiazolidinecarboxamide 1,1-dioxide (E20);
2-ethyl-N-{[4-fluoro-2-(trifluoromethyl)phenyl]methyl}-3-isothiazolidinecarboxamide 1,1-dioxide (E21);
N-[(2-chloro-4-fluorophenyl)methyl]-2-(1-methylethyl)-3-isothiazolidinecarboxamide 1,1-dioxide (E22);
2-(1-methylethyl)-N-[(2,3,4-trifluorophenyl)methyl]-3-isothiazolidinecarboxamide 1,1-dioxide (E23);
N-[(2,4-dichlorophenyl)methyl]-2-ethyl-3-isothiazolidinecarboxamide 1,1-dioxide (E24);
2-(2-methylpropyl)-N-[(2,3,4-trifluorophenyl)methyl]-3-isothiazolidinecarboxamide 1,1-dioxide (E25);
N-[(2-chloro-6-methylphenyl)methyl]-2-(1-methylethyl)-3-isothiazolidinecarboxamide 1,1-dioxide (E26);
N-[(2,4-dichlorophenyl)methyl]-2-(1-methylethyl)-3-isothiazolidinecarboxamide 1,1-dioxide (E27);
N-[(2-chloro-6-methylphenyl)methyl]-2-ethyl-3-isothiazolidinecarboxamide 1,1-dioxide (E28);
N-{[2-chloro-3-(trifluoromethyl)phenyl]methyl}-2-methyl-3-isothiazolidinecarboxamide 1,1-dioxide (E29);
N-[(2-chloro-3,4-difluorophenyl)methyl]-2-methyl-3-isothiazolidinecarboxamide 1,1-dioxide (E30);
N-[(2-chloro-3,4-difluorophenyl)methyl]-2-ethyl-3-isothiazolidinecarboxamide 1,1-dioxide (E31);
N-{[2-chloro-3-(trifluoromethyl)phenyl]methyl}-2-ethyl-3-isothiazolidinecarboxamide 1,1-dioxide (E32);
2-ethyl-N-{[4-fluoro-2-(trifluoromethyl)phenyl]methyl}tetrahydro-2H-1,2-thiazine-3-carboxamide 1,1-dioxide (E33);
N-[(2-chloro-4-fluorophenyl)methyl]-2-ethyltetrahydro-2H-1,2-thiazine-3-carboxamide 1,1-dioxide (E34);
N-[(2,4-dichlorophenyl)methyl]-2-methyltetrahydro-2H-1,2-thiazine-3-carboxamide 1,1-dioxide (E35);
N-[(2,4-dichlorophenyl)methyl]-2-ethyltetrahydro-2H-1,2-thiazine-3-carboxamide 1,1-dioxide (E36);
N-[(2-chloro-4-fluorophenyl)methyl]-2-methyltetrahydro-2H-1,2-thiazine-3-carboxamide 1,1-dioxide (E37);
N-[(3-chloro-2-methylphenyl)methyl]-2-ethyltetrahydro-2H-1,2-thiazine-3-carboxamide 1,1-dioxide (E38);
N-[(2-chlorophenyl)methyl]-2-ethyltetrahydro-2H-1,2-thiazine-3-carboxamide 1,1-dioxide (E39);
N-[(2,4-dichlorophenyl)methyl]-2-(1-methylethyl)tetrahydro-2H-1,2-thiazine-3-carboxamide 1,1-dioxide (E40);
N-[(2-chloro-6-methylphenyl)methyl]-2-(1-methylethyl)tetrahydro-2H-1,2-thiazine-3-carboxamide 1,1-dioxide (E41);
N-[(3-chloro-2-methylphenyl)methyl]-2-(1-methylethyl)tetrahydro-2H-1,2-thiazine-3-carboxamide 1,1-dioxide (E42);
N-[(2-chloro-4-fluorophenyl)methyl]-2-(1-methylethyl)tetrahydro-2H-1,2-thiazine-3-carboxamide 1,1-dioxide (E43);
N-[(2-chlorophenyl)methyl]-2-(1-methylethyl)tetrahydro-2H-1,2-thiazine-3-carboxamide 1,1-dioxide (E44);
N-[(2-chloro-6-methylphenyl)methyl]-2-ethyltetrahydro-2H-1,2-thiazine-3-carboxamide 1,1-dioxide (E45);
2-(1-methylethyl)-N-[(2,3,4-trifluorophenyl)methyl]tetrahydro-2H-1,2-thiazine-3-carboxamide 1,1-dioxide (E46);
N-{[4-fluoro-2-(trifluoromethyl)phenyl]methyl}-2-(1-methylethyl)tetrahydro-2H-1,2-thiazine-3-carboxamide 1,1-dioxide (E47);
N-{[4-fluoro-2-(trifluoromethyl)phenyl]methyl}-2-methyltetrahydro-2H-1,2-thiazine-3-carboxamide 1,1-dioxide (E48);
N-[(3-chloro-2-methylphenyl)methyl]-2-methyltetrahydro-2H-1,2-thiazine-3-carboxamide 1,1-dioxide (E49);
N-[(2-chlorophenyl)methyl]-2-methyltetrahydro-2H-1,2-thiazine-3-carboxamide 1,1-dioxide (E50);
2-ethyl-N-[(2,3,4-trifluorophenyl)methyl]tetrahydro-2H-1,2-thiazine-3-carboxamide 1,1-dioxide (E51);
N-[(2-chloro-6-methylphenyl)methyl]-2-methyltetrahydro-2H-1,2-thiazine-3-carboxamide 1,1-dioxide (E52); or
2-methyl-N-[(2,3,4-trifluorophenyl)methyl]tetrahydro-2H-1,2-thiazine-3-carboxamide 1,1-dioxide (E53).

In one preferable embodiment of the invention, there is provided a compound of formula (1) [in which n represents 1 and X represents —CH₂—], or a pharmaceutically acceptable salt thereof, which is:
N-{[4-fluoro-2-(trifluoromethyl)phenyl]methyl}-2-(2-methylpropyl)-3-isothiazolidinecarboxamide 1,1-dioxide (E1);
N-[(2-chloro-4-fluorophenyl)methyl]-2-(2-methylpropyl)-3-isothiazolidinecarboxamide 1,1-dioxide (E2);
N-[(2-chloro-4-fluorophenyl)methyl]-2-methyl-3-isothiazolidinecarboxamide 1,1-dioxide (E3);
N-[(2,4-dichlorophenyl)methyl]-2-(2-methylpropyl)-3-isothiazolidinecarboxamide 1,1-dioxide (E4);
N-[(2-chloro-6-methylphenyl)methyl]-2-(2-methylpropyl)-3-isothiazolidinecarboxamide 1,1-dioxide (E5);
N-[(2-chlorophenyl)methyl]-2-(1-methylethyl)-3-isothiazolidinecarboxamide 1,1-dioxide (E6);
N-[(2-chlorophenyl)methyl]-2-ethyl-3-isothiazolidinecarboxamide 1,1-dioxide (E7);
N-{[4-fluoro-2-(trifluoromethyl)phenyl]methyl}-2-(1-methylethyl)-3-isothiazolidinecarboxamide 1,1-dioxide (E8);
N-[(2-chlorophenyl)methyl]-2-methyl-3-isothiazolidinecarboxamide 1,1-dioxide (E9);
N-{[4-fluoro-2-(trifluoromethyl)phenyl]methyl}-2-methyl-3-isothiazolidinecarboxamide 1,1-dioxide (E10);
N-[(2-chlorophenyl)methyl]-2-(2-methylpropyl)-3-isothiazolidinecarboxamide 1,1-dioxide (E11);
N-[(2-chloro-6-methylphenyl)methyl]-2-methyl-3-isothiazolidinecarboxamide 1,1-dioxide (E12);
N-[(2,4-dichlorophenyl)methyl]-2-methyl-3-isothiazolidinecarboxamide 1,1-dioxide (E13);
2-ethyl-N-[(2,3,4-trifluorophenyl)methyl]-3-isothiazolidinecarboxamide 1,1-dioxide (E14);
N-[(2-chloro-4-fluorophenyl)methyl]-2-ethyl-3-isothiazolidinecarboxamide 1,1-dioxide (E15);
2-methyl-N-[(2,3,4-trifluorophenyl)methyl]-3-isothiazolidinecarboxamide 1,1-dioxide (E16);
N-[(3-chloro-2-methylphenyl)methyl]-2-methyl-3-isothiazolidinecarboxamide 1,1-dioxide (E17);
N-[(3-chloro-2-methylphenyl)methyl]-2-(1-methylethyl)-3-isothiazolidinecarboxamide 1,1-dioxide (E18);
N-[(3-chloro-2-methylphenyl)methyl]-2-(2-methylpropyl)-3-isothiazolidinecarboxamide 1,1-dioxide (E19);
N-[(3-chloro-2-methylphenyl)methyl]-2-ethyl-3-isothiazolidinecarboxamide 1,1-dioxide (E20);
2-ethyl-N-{[4-fluoro-2-(trifluoromethyl)phenyl]methyl}-3-isothiazolidinecarboxamide 1,1-dioxide (E21);
N-[(2-chloro-4-fluorophenyl)methyl]-2-(1-methylethyl)-3-isothiazolidinecarboxamide 1,1-dioxide (E22);
2-(1-methylethyl)-N-[(2,3,4-trifluorophenyl)methyl]-3-isothiazolidinecarboxamide 1,1-dioxide (E23);
N-[(2,4-dichlorophenyl)methyl]-2-ethyl-3-isothiazolidinecarboxamide 1,1-dioxide (E24);
2-(2-methylpropyl)-N-[(2,3,4-trifluorophenyl)methyl]-3-isothiazolidinecarboxamide 1,1-dioxide (E25);
N-[(2-chloro-6-methylphenyl)methyl]-2-(1-methylethyl)-3-isothiazolidinecarboxamide 1,1-dioxide (E26);
N-[(2,4-dichlorophenyl)methyl]-2-(1-methylethyl)-3-isothiazolidinecarboxamide 1,1-dioxide (E27);
N-[(2-chloro-6-methylphenyl)methyl]-2-ethyl-3-isothiazolidinecarboxamide 1,1-dioxide (E28);
N-{[2-chloro-3-(trifluoromethyl)phenyl]methyl}-2-methyl-3-isothiazolidinecarboxamide 1,1-dioxide (E29);
N-[(2-chloro-3,4-difluorophenyl)methyl]-2-methyl-3-isothiazolidinecarboxamide 1,1-dioxide (E30);
N-[(2-chloro-3,4-difluorophenyl)methyl]-2-ethyl-3-isothiazolidinecarboxamide 1,1-dioxide (E31); or
N-{[2-chloro-3-(trifluoromethyl)phenyl]methyl}-2-ethyl-3-isothiazolidinecarboxamide 1,1-dioxide (E32).

In one particular embodiment, these above-listed compounds (in which n represents 1, and X represents —CH₂—, e.g. from Examples 1 to 32), or pharmaceutically acceptable salts thereof, are prepared from L-homocystine (see e.g. Examples section herein). Therefore these above-listed compounds or salts (in which n represents 1, and X represents —CH₂—) can be, in one particular embodiment, in a

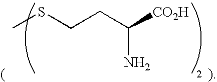

form prepared or obtainable from L-homocystine

A particular aspect of the present invention provides a compound of formula (1A) or a pharmaceutically acceptable salt thereof:

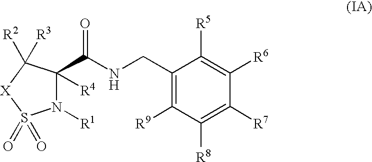

wherein:
R¹ represents C₁₋₄ alkyl optionally substituted with 1, 2 or 3 halogen (e.g. fluorine) atoms;
and X, n, R², R³, R⁴, R⁵, R⁶, R⁷, R⁸, R⁹, R¹⁰, and R¹¹ are as defined herein for the compound of formula (1) or the pharmaceutically acceptable salt thereof, and wherein more than 50% (e.g. more than 70%, in particular more than 90%, such as more than 95%) by molarity of the compound of formula (1A) or the pharmaceutically acceptable salt thereof has the indicated stereochemistry at the ring-carbon atom bonded to R⁴.

In a particular embodiment, the compound of formula (1A) or the pharmaceutically acceptable salt thereof has an enantiomeric excess of greater than 70% (e.g. more than 80%, in particular more than 90%) with respect to the indicated stereochemistry at the ring-carbon atom bonded to R⁴.

In a particular embodiment, in a compound of formula (1A) or a salt thereof, R¹ represents unsubstituted C₁₋₄ alkyl such as unsubstituted C₁₋₃ alkyl; more particularly methyl, ethyl, or isopropyl; most particularly methyl or ethyl.

In a particular embodiment, in a compound of formula (1A) or a salt thereof, R², R³ and R⁴ each represent hydrogen.

All embodiments, e.g. particular or preferable features or aspects, of the invention (e.g. embodiments of the compound or salt of the invention and/or of pharmaceutical compositions and/or uses or methods of treatment or combinations thereof, et al.) which are disclosed herein in relation to a compound of formula (1) or a salt thereof, are also hereby disclosed and contemplated in relation to a compound of formula (1A) or a salt thereof, to the extent appropriate or possible, with all necessary changes having been made to the wording.

An alternative particular aspect of the invention provides a compound of formula (1) or a pharmaceutically acceptable salt thereof, as disclosed herein, wherein the compound or salt is substantially racemic (e.g. racemic) at the ring-carbon atom bonded to R⁴.

Antagonists of P2X7 may be useful in preventing, treating, or ameliorating a variety of pain states (e.g. neuropathic pain, chronic inflammatory pain, or visceral pain), inflammation (e.g. rheumatoid arthritis or osteoarthritis), and neurodegenerative diseases, in particular Alzheimer's disease. P2X7 antagonists may constitute useful therapeutic agents in the management of rheumatoid arthritis and inflammatory bowel disease.

Compounds or salts of the present invention which modulate P2X7 receptor function and are capable of antagonizing the effects of ATP at the P2X7 receptor (“P2X7 receptor antagonists”) may be competitive antagonists, inverse agonists, or negative allosteric modulators of P2X7 receptor function.

Certain compounds of formula (1) may in some circumstances form acid addition salts thereof. It will be appreciated that for use in medicine compounds of formula (1) may be used as salts, in which case the salts should be pharmaceutically acceptable. Pharmaceutically acceptable salts include those described by Berge, Bighley and Monkhouse, J. Pharm. Sci., 1977, 66, 1-19.

When a compound of formula (1) is basic compounds of formula (1), in one embodiment a pharmaceutically acceptable salt is formed from a pharmaceutically acceptable acid such as an inorganic or organic acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one embodiment, the pharmaceutically acceptable acid is benzenesulfonic, camphorsulfonic, ethanesulfonic, hydrobromic, hydrochloric, methanesulfonic, nitric, phosphoric, sulfuric, or p-toluenesulfonic acid.

Examples of pharmaceutically acceptable salts include those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The compounds of formula (1) or pharmaceutically acceptable salts thereof may be prepared in crystalline or non-crystalline form (e.g. in crystalline or amorphous solid form), and, in particular if crystalline, may optionally be solvated, e.g. as the hydrate. This invention includes within its scope solvates (e.g. hydrates) of compounds of formula (1) or pharmaceutically acceptable salts thereof, for example stoichiometric solvates (e.g. hydrates); as well as compounds or salts thereof containing variable amounts of solvent (e.g. water).

Certain compounds of formula (1) or salts thereof may be capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The subject invention also includes isotopically-labelled compounds, which are identical to those recited in formula (1) or salts thereof, but for the fact that one or more atoms are replaced by an atom having an atomic mass or mass number different from the atomic mass or mass number most commonly found in nature. Examples of isotopes that can be incorporated into compounds or salts of the invention include isotopes of hydrogen, carbon, nitrogen, oxygen, phosphorous, fluorine, iodine, and chlorine, such as 3H, 11C, 14C, 18F, 1231 and 1251.

Compounds of the present invention and pharmaceutically acceptable salts of said compounds that contain the aforementioned isotopes and/or other isotopes of other atoms are within the scope of the present invention. Isotopically-labelled compounds or salts of the present invention, for example those into which radioactive isotopes such as 3H, 14C are incorporated, are potentially useful in drug and/or substrate tissue distribution assays. Tritiated, i.e., 3H, and carbon-14, i.e., 14C, isotopes are for example optionally chosen for their (in some cases) ease of preparation and/or detectability. 11C and 8F isotopes can sometimes be useful in PET (positron emission tomography), and 1251 isotopes can sometimes be useful in SPECT (single photon emission computerized tomography). PET and SPECT can sometimes be useful in brain imaging. Further, substitution with heavier isotopes such as deuterium, i.e., 2H, can sometimes afford certain effects resulting from greater metabolic stability, for example increased in vivo half-life or reduced dosage requirements and, hence, may be chosen in some circumstances. Isotopically labelled compounds of formula (1) or salts thereof of this invention are in one embodiment and in some cases prepared by carrying out the procedures disclosed in the Schemes and/or in the Examples below, by substituting an available isotopically labelled reagent for a non-isotopically labelled reagent.

A further particular aspect of the invention provides a compound of formula (1) or a pharmaceutically acceptable salt thereof which is not a radioactive isotopically-labelled compound or salt. In a particular embodiment, the compound or salt is not an isotopically-labelled compound or salt.

### Preparation of Compounds

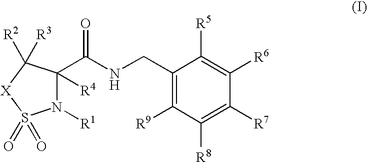

Compounds of formula (1), wherein the variables are as defined herein, and pharmaceutically acceptable salts thereof may be prepared by the methodology described hereinafter, constituting a further aspect of this invention.

According to a further aspect of the invention, a process for preparing a compound of formula (1) or a pharmaceutically acceptable salt thereof comprises step (a), (b) or (c), as described below;

and optionally preparing a pharmaceutically acceptable salt of the compound.
(a) Coupling of a carboxylic acid of formula (2) (or an activated derivative thereof) with an amine of formula (3) (see Scheme 1), wherein X, R¹, R², R³, R⁴, R⁵, R⁶, R⁷, R⁸, and R⁹ are as defined above. Compounds (2) and (3) are optionally protected.

The coupling of an acid of formula (2) and an amine of formula (3) typically comprises the use of an activating agent, such as N-(3-dimethylaminopropyl)-N′-ethylcarbodiimide hydrochloride or polymer-supported carbodiimide, 1-hydroxybenzotriazole (HOBT) or 1-hydroxy-7-azabenzotriazole (HOAt), and optionally a suitable base such as a tertiary alkylamine (e.g. diisopropylethylamine, N-ethyl morpholine, triethylamine) or pyridine, in a suitable solvent such as DMF and/or dichloromethane and at a suitable temperature e.g. between 0° C. and room temperature. Alternatively the coupling of (2) and (3) may be accomplished by treatment with O-(7-azabenzotriazol-1-yl)-N,N, N′,N′-tetramethyluronium hexafluorophosphate and a suitable tertiary alkylamine such as diisopropylamine in a suitable solvent such as dimethylformamide at a suitable temperature such as room temperature. Alternatively, the compound of formula (2) may be employed as an activated derivative (e.g. acid chloride, mixed anhydride, active ester (e.g. O-acyl-isourea)), and under such circumstances process (a) typically comprises treatment of said activated derivative with an amine (Ogliaruso, M. A.; Wolfe, J. F. in The Chemistry of Functional Groups (Ed. Patai, S.) Suppl. B: The Chemistry of Acid Derivatives, Pt. 1 (John Wiley and Sons, 1979), pp 442-8; Beckwith, A. L. J. in The Chemistry of Functional Groups (Ed. Patai, S.) Suppl. B: The Chemistry of Amides (Ed. Zabricky, J.)(John Wiley and Sons, 1970), pp 73 ff).

(b) Deprotecting a compound of formula (1) which is protected. Examples of protecting groups and the means for their removal can be found in T. W. Greene and P. G. M. Wuts ‘Protective Groups in Organic Synthesis’ (Wiley-Interscience, 4th Ed. 2006).
(c) Interconversion of compounds of formula (1) to other compounds of formula (1). Examples of conventional interconversion procedures include epimerisation, oxidation, reduction, alkylation, aromatic substitution, nucleophilic substitution, amide coupling and ester hydrolysis.

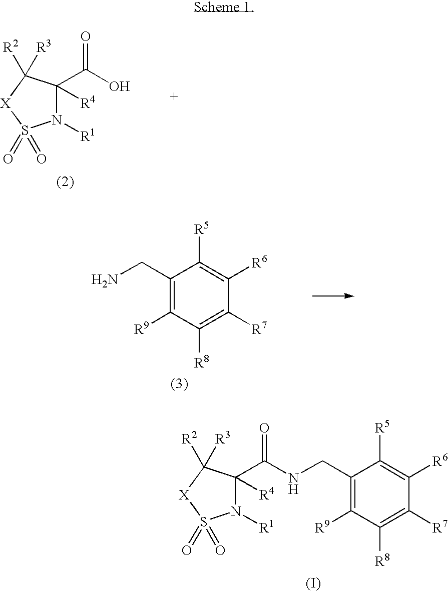

Representative methods for the preparation of compounds of formula (2) are shown in Schemes 2-3 below. Some of these transformations and/or analogous methods are described in the chemical literature e.g. G. Luisi & F. Pinnen, Arch. Pharm. (Weinheim) 326, 139-141 (1993); R. J. Chemey, et. al., J. Med. Chem., 47, 2981-2983, (2004).

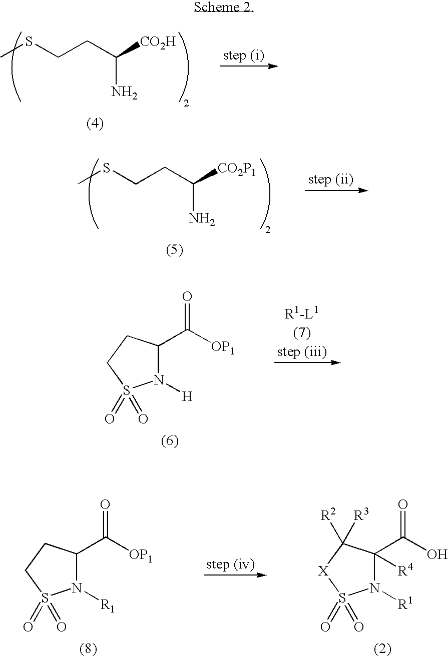

wherein X, R¹, R², R³, and R⁴ are as defined above. P¹ represents a suitable carboxylate protecting group such as C₁₋₆ alkyl. L¹ represents a suitable leaving group such as halogen (e.g. bromine, iodine).

Step (i) typically comprises standard protection of the carboxylic acid (4) (L-homocystine) as the corresponding carboxylic ester for example by treatment with thionyl chloride in a suitable solvent such as methanol at a suitable temperature such as between 0° C. and room temperature.

Step (ii) typically comprises oxidation of compound (5) with a reagent such as chlorine followed by treatment with a suitable base such as triethylamine at a suitable temperature, such as between 0° C. and room temperature and, in a suitable solvent, such as a mixture of ethanol and chloroform, to afford compound (6).

Step (iii) typically comprises treatment of compound (6) with a suitable base such as potassium carbonate and an alkylating agent (7) such as an alkyl halide (e.g. methyl iodide) at a suitable temperature, such as between room temperature and 70° C. and, in a suitable solvent, such as dimethylformamide.

Deprotection step (iv) typically comprises a standard procedure for conversion of a carboxylic ester (8) to an acid (2), such as use of an appropriate hydroxide salt (e.g. lithium hydroxide) in an appropriate solvent such as a mixture of tetrahydrofuran and water at a suitable temperature such as room temperature.

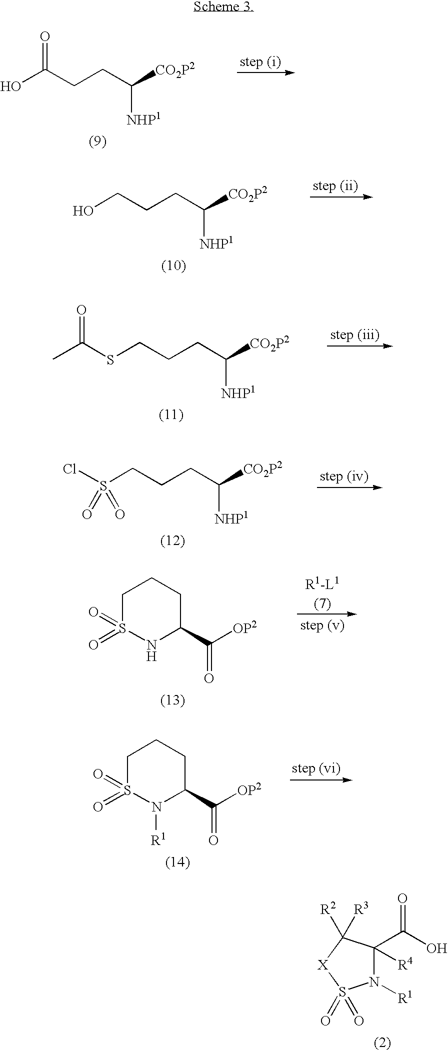

wherein X, R¹, R², R³, and R⁴ are as defined above. P¹ represents a suitable amino protecting group such as tert-butyloxy carbonyl and P² represents a suitable carboxylate protecting group such as C₁₋₆ alkyl. L¹ represents a suitable leaving group such as halogen (e.g. bromine, iodine).

Step (i) typically comprises a standard method for reduction of a carboxylic acid (9) to the corresponding alcohol (10) for example by formation of a mixed anhydride using a suitable chloroformate such as ethyl chloroformate and a suitable base such as triethylamine and subsequent reduction using a suitable reducing agent such as sodium borohydride in a suitable solvent such as tetrahydrofuran at a suitable temperature such as between 0° C. and room temperature.

Step (ii) typically comprises treatment of compound (10) with thioacetic acid and triphenylphosphine and a suitable azodicarboxylate reagent such as diisopropyl azodicarboxylate at a suitable temperature, such as between 0° C. and room temperature in a suitable solvent such as tetrahydrofuran to afford compound (11).

Step (iii) typically comprises oxidation of compound (11) with a suitable reagent such as chlorine at a suitable temperature, such as 3° C. and, in a suitable solvent, such as water.

Deprotection of (12) and cyclisation to give (13), step (iv), typically comprises a standard procedure for deprotection of a carbamate protected amino group such as use of an appropriate acid (e.g. hydrogen chloride) in an appropriate solvent such as a mixture of tetrahydrofuran and dioxane at a suitable temperature such as 0° C. followed by treatment with a suitable base such as triethylamine in a suitable solvent such as chloroform at a suitable temperature such as between −5° C. and room temperature.

Step (v) typically comprises treatment of compound (13) with a suitable base such as potassium carbonate and an alkylating agent (7) such as an alkyl halide (e.g. methyl iodide) at a suitable temperature, such as between room temperature and 70° C. and, in a suitable solvent, such as dimethylformamide.

Deprotection step (vi) typically comprises a standard procedure for conversion of a carboxylic ester (14) to an acid (2), such as use of an appropriate acid (e.g. trifluoroacetic acid) in an appropriate solvent such as dichloromethane at a suitable temperature such as between 0° C. and room temperature.

Compounds of the general formulae (3), (4), (7) and (9) are typically either available from commercial sources or can be prepared by a person skilled in the art using methods described in the chemical literature (or using analogous methods).

Where relevant, pharmaceutically acceptable salts may be prepared by reaction with the appropriate acid or acid derivative.

### Clinical Indications, Pharmaceutical Compositions, and Dosages

It is believed that, as the compounds or pharmaceutically acceptable salts of the present invention modulate P2X7 receptor function and are capable of antagonizing the effects of ATP at the P2X7 receptor (“P2X7 receptor antagonists”); they may be useful in the treatment of pain; such as acute pain, chronic pain, chronic articular pain, musculoskeletal pain, neuropathic pain, inflammatory pain, visceral pain, pain associated with cancer, pain associated with migraine, tension headache or cluster headaches, pain associated with functional bowel disorders, lower back and/or neck pain, pain associated with sprains and/or strains, sympathetically maintained pain; myositis, pain associated with influenza or other viral infections such as the common cold, pain associated with rheumatic fever, pain associated with myocardial ischemia, post operative pain, cancer chemotherapy, headache, toothache, or dysmenorrhea.

The chronic articular pain condition can be rheumatoid arthritis, osteoarthritis, rheumatoid spondylitis (ankylosing spondylitis), gouty arthritis or juvenile arthritis.

The inflammatory pain condition can be rheumatoid arthritis, osteoarthritis, rheumatoid spondylitis (ankylosing spondylitis) or fibromyalgia.

In particular, the compounds of formula (1) or pharmaceutically acceptable salts thereof may be useful in the treatment or prevention (treatment or prophylaxis) of pain (e.g. inflammatory pain) in arthritis, such as pain (e.g. inflammatory pain) in rheumatoid arthritis or osteoarthritis.

Pain associated with functional bowel disorders includes non-ulcer dyspepsia, non-cardiac chest pain and irritable bowel syndrome.

The neuropathic pain condition can be: diabetic neuropathy (e.g. painful diabetic neuropathy), sciatica, non-specific lower back pain, trigeminal neuralgia, multiple sclerosis pain, fibromyalgia, HIV-related neuropathy, post-herpetic neuralgia, trigeminal neuralgia, or lumbar radiculopathy; or pain resulting from physical trauma, amputation, phantom limb syndrome, spinal surgery, cancer, toxins or chronic inflammatory conditions. Alternatively, the neuropathic pain condition can be pain associated with normally non-painful sensations such as “pins and needles” (paraesthesias and/or dysesthesias), increased sensitivity to touch (hyperesthesia), painful sensation following innocuous stimulation (dynamic, static, thermal or cold allodynia), increased sensitivity to noxious stimuli (thermal, cold, or mechanical hyperalgesia), continuing pain sensation after removal of the stimulation (hyperpathia), or an absence of or deficit in selective sensory pathways (hypoalgesia).

The acute pain condition can be post-surgical pain or dysmenorrhea (e.g. primary dysmenorrhea).

Other conditions which could potentially be treated by compounds or salts of the present invention include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) such as cyclooxygenase-2 (COX-2) inhibitors, cardiovascular diseases; neurodegenerative diseases and neurodegeneration, neurodegeneration following trauma, tinnitus, dependence on a dependence-inducing agent such as opiods (e.g. morphine), CNS (central nervous system) depressants (e.g. ethanol), psychostimulants (e.g. cocaine) or nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence. Depression and alcoholism could potentially also be treated by compounds or salts of the present invention.

Inflammation and the inflammatory conditions associated with said inflammation include arthritis (in particular rheumatoid arthritis or osteoarthritis), skin conditions (e.g. sunburn, burns, eczema, dermatitis, allergic dermatitis, psoriasis), meningitis, ophthalmic diseases such as glaucoma, retinitis, retinopathies, uveitis and of acute injury to the eye tissue (e.g. conjunctivitis), inflammatory lung disorders (e.g. asthma, allergic rhinitis, respiratory distress syndrome, pigeon fancier's disease, farmer's lung, chronic obstructive pulmonary disease (COPD, which includes bronchitis and/or emphysema), or airways hyperresponsiveness); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, or gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome. Inflammation or an inflammatory condition associated with said inflammation can in particular be arthritis (e.g. rheumatoid arthritis or osteoarthritis).

Immunological diseases include autoimmune diseases, immunological deficiency diseases or organ transplantation.

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

A bone disease characterised by abnormal bone metabolism or resorbtion may particular be rheumatoid arthritis or osteoarthritis, for potential treatment by compounds or pharmaceutically acceptable salts of the present invention.

Cardiovascular diseases include hypertension or myocardiac ischemia; atherosclerosis; functional or organic venous insufficiency; varicose therapy; haemorrhoids; and shock states associated with a marked drop in arterial pressure (e.g. septic shock).

Neurodegenerative diseases include dementia, particularly degenerative dementia (such as senile dementia, dementia with Lewy bodies, Alzheimer's disease, Pick's disease, Huntingdon's chorea, Parkinson's disease and Creutzfeldt-Jakob disease, Amyotrophic Lateral Sclerosis (ALS) or motor neuron disease; in particular Alzheimer's disease); vascular dementia (including multi-infarct dementia); as well as dementia associated with intracranial space occupying lesions; trauma; infections and related conditions (including HIV infection, meningitis and shingles); metabolism; toxins; anoxia and vitamin deficiency; and mild cognitive impairment e.g. associated with ageing, particularly age associated memory impairment.

The neurodegenerative disease, e.g. to be treated by the compound of formula (1) or salt thereof, can for example be degenerative dementia (in particular Alzheimer's disease), vascular dementia (in particular multi-infarct dementia), or mild cognitive impairment (MC1) e.g. MC1 associated with ageing such as age associated memory impairment.

The compounds of formula (1) or pharmaceutically acceptable salts thereof may also be useful for neuroprotection and in the treatment of neurodegeneration following trauma such as stroke, cardiac arrest, pulmonary bypass, traumatic brain injury, spinal cord injury or the like.

The compounds or pharmaceutically acceptable salts of the present invention may also be useful in the treatment of malignant cell growth and/or metastasis, and myoblastic leukaemia.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, glomerulonephritis, particularly mesangial proliferative glomerulonephritis and nephritic syndrome.

It is to be understood that reference to treatment includes both treatment of established symptoms and prophylactic treatment, unless explicitly stated otherwise.

According to a further aspect of the invention, we therefore provide a compound of formula (1) or a pharmaceutically acceptable salt thereof for use in human or veterinary medicine and/or for use in therapy.

According to another aspect of the invention, we provide a compound of formula (1) or a pharmaceutically acceptable salt thereof for use in the treatment or prevention (e.g. treatment) of a condition which is mediated by P2X7 receptors, for example a condition or disease disclosed herein (in particular pain, inflammation such as rheumatoid arthritis or osteoarthritis, or a neurodegenerative disease; more particularly pain such as inflammatory pain, neuropathic pain or visceral pain, or rheumatoid arthritis or osteoarthritis); e.g. in a mammal such as a human or rodent e.g. human or rat e.g. human.

According to a further aspect of the invention, we provide a method of treating a human or animal (e.g. rodent e.g. rat) subject, for example a human subject, suffering from a condition which is mediated by P2X7 receptors, for example a condition or disease disclosed herein (in particular pain, inflammation such as rheumatoid arthritis or osteoarthritis, or a neurodegenerative disease; more particularly pain such as inflammatory pain, neuropathic pain or visceral pain, or rheumatoid arthritis or osteoarthritis), which comprises administering to said subject an effective amount of a compound of formula (1) or a pharmaceutically acceptable salt thereof.

According to a further aspect of the invention we provide a method of treating a human or animal (e.g. rodent e.g. rat) subject, for example a human subject, suffering from pain, inflammation (e.g. rheumatoid arthritis or osteoarthritis), or a neurodegenerative disease (more particularly pain such as inflammatory pain, neuropathic pain or visceral pain, or rheumatoid arthritis or osteoarthritis), which method comprises administering to said subject an effective amount of a compound of formula (1) or a pharmaceutically acceptable salt thereof.

According to a yet further aspect of the invention we provide a method of treating a human or animal (e.g. rodent e.g. rat) subject, for example a human subject, suffering from inflammatory pain, neuropathic pain or visceral pain (e.g. pain, such as inflammatory pain, in arthritis (e.g. rheumatoid arthritis or osteoarthritis)) which method comprises administering to said subject an effective amount of a compound of formula (1) or a pharmaceutically acceptable salt thereof.

According to a further aspect of the invention we provide a method of treating a subject, for example a human subject, suffering from Alzheimer's disease which method comprises administering to said subject an effective amount of a compound of formula (1) or a pharmaceutically acceptable salt thereof.

According to another aspect of the invention, we provide the use of a compound of formula (1) or a pharmaceutically acceptable salt thereof for the manufacture of a medicament for the treatment or prevention (e.g. treatment) of a condition which is mediated by the action of P2X7 receptors, for example a condition or disease disclosed herein (in particular pain, inflammation such as rheumatoid arthritis or osteoarthritis, or a neurodegenerative disease; more particularly pain such as inflammatory pain, neuropathic pain or visceral pain); e.g. in a mammal such as a human or rodent e.g. human or rat e.g. human.

According to another aspect of the invention we provide the use of a compound of formula (1) or a pharmaceutically acceptable salt thereof for the manufacture of a medicament for the treatment or prevention (e.g. treatment) of pain (e.g. inflammatory pain, neuropathic pain or visceral pain), inflammation (e.g. rheumatoid arthritis or osteoarthritis), or a neurodegenerative disease (more particularly: pain such as inflammatory pain, neuropathic pain or visceral pain, or rheumatoid arthritis or osteoarthritis); e.g. in a mammal such as a human or rodent e.g. human or rat e.g. human.

According to another aspect of the invention we provide the use of a compound of formula (1) or a pharmaceutically acceptable salt thereof for the manufacture of a medicament for the treatment or prevention (e.g. treatment) of inflammatory pain, neuropathic pain or visceral pain (in particular inflammatory pain or neuropathic pain; such as inflammatory pain in arthritis such as rheumatoid arthritis or osteoarthritis); e.g. in a mammal such as a human or rodent e.g. human or rat e.g. human.

In one aspect of the invention we provide the use of a compound of formula (1) or a pharmaceutically acceptable salt thereof for the manufacture of a medicament for the treatment or prevention (e.g. treatment) of Alzheimer's disease; e.g. in a mammal such as a human or rodent e.g. human or rat e.g. human.

In order to use a compound of formula (1) or a pharmaceutically acceptable salt thereof for the treatment of humans and/or other mammals it can optionally be formulated in accordance with pharmaceutical practice as a pharmaceutical composition. Therefore in another aspect of the invention there is provided a pharmaceutical composition comprising a compound of formula (1), or a pharmaceutically acceptable salt thereof, adapted for use in human or veterinary medicine.

In order to use a compounds of formula (1) or a pharmaceutically acceptable salt thereof in therapy, it will normally be formulated into a pharmaceutical composition in accordance with pharmaceutical practice. The present invention also provides a pharmaceutical composition, which comprises a compound of formula (1), or a pharmaceutically acceptable salt thereof, and a pharmaceutically acceptable carrier or excipient.

The pharmaceutical composition may be for use in a method of treatment or in a use or in a treatment or prevention, as described herein.

A pharmaceutical composition of the invention, which may be prepared by admixture, for example at ambient temperature and/or atmospheric pressure, is usually adapted for oral, parenteral or rectal administration. As such, the pharmaceutical composition may be in the form of a tablet, a capsule, a oral liquid preparation, a powder, a granule, a lozenge, a reconstitutable powder, an injectable or infusable solution or suspension, or a suppository.

An orally administrable pharmaceutical composition is generally preferred.

Tablets and capsules for oral administration may be in unit dose form, and may contain one or more excipients, such as a binding agent (e.g. hydroxypropylmethylcellulose or povidone), a filler (e.g. lactose and/or microcrystalline cellulose), a lubricant e.g. a tabletting lubricant (e.g. magnesium stearate or calcium stearate), a disintegrant (e.g. a tablet disintegrant such as sodium starch glycolate or croscarmellose sodium), and/or an acceptable wetting agent. The tablets may be coated e.g. according to methods known in pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain additive(s) such as a suspending agent(s), an emulsifying agent(s), a non-aqueous vehicle(s) (such as an edible oil), and/or a preservative(s), and/or, if desired, a flavouring(s) or colourant(s).

For parenteral administration, fluid unit dosage forms are typically prepared utilising a compound of the invention or pharmaceutically acceptable salt thereof and a sterile vehicle. In one embodiment, the compound or salt, depending on the vehicle and concentration used, is either suspended or dissolved in the vehicle. In preparing solutions, the compound or salt can e.g. be dissolved for injection and filter sterilised before filling into a suitable vial or ampoule and sealing. In one embodiment, an adjuvant(s) such as a local anaesthetic, a preservative(s) and/or a buffering agent(s) is or are dissolved in the vehicle. To enhance the stability, the composition can for example be frozen after filling into the vial and the water removed under vacuum. Parenteral suspensions are typically prepared in substantially the same manner, except that the compound or salt is typically suspended in the vehicle instead of being dissolved, and sterilization is not usually accomplished by filtration. The compound or salt can be sterilised, e.g. by exposure to ethylene oxide, before suspension in a sterile vehicle. In one embodiment, a surfactant or wetting agent is included in the composition, e.g. to facilitate uniform distribution of the compound or salt of the invention.

In one embodiment, the composition contains from 0.1% to 99% (by weight of the composition), in particular from 0.1 to 60% or 1 to 60% or 10 to 60% by weight, of the active material (the compound or pharmaceutically acceptable salt of the invention), e.g. depending on the method of administration. The carrier(s) and/or excipient(s) contained in the composition can for example be present in from 1% to 99.9%, e.g. from 10% to 99%, by weight of the composition; and/or in an amount of from 20 mg to 2000 mg such as 50 mg to 1000 mg per unit dose of the composition.

The dose of the compound or pharmaceutically acceptable salt thereof, e.g. for use in the treatment or prevention (e.g. treatment) of the hereinmentioned disorders/diseases/conditions, may vary in the usual way with the seriousness of the disorders, the weight of the sufferer, and/or other similar factors. However, as a general guide, in one embodiment a unit dose of 0.05 to 2000 mg or 0.05 to 1000 mg, for example 0.05 to 200 mg, such as 20 to 40 mg, of the compound or pharmaceutically acceptable salt of the invention (measured as the compound), may be used, e.g. in a pharmaceutical composition. In one embodiment, such a unit dose is for administration once a day e.g. to a mammal such as a human; alternatively such a unit dose may be for administration more than once (e.g. twice or three times) a day e.g. to a mammal such as a human. Such therapy may extend for a number of days, weeks, months or years.

### Combinations

Compounds of formula (1) or pharmaceutically acceptable salts thereof may be used in combination with other (further) therapeutic agent(s), for example medicaments claimed to be useful in the treatment or prevention (e.g. treatment) of the above mentioned disorders.

Examples of such further therapeutic agent(s) may include a β2-agonist (also known as β2 adrenoceptor agonists; e.g. formoterol) and/or a corticosteroid (e.g. budesonide, fluticasone (e.g. as propionate or furoate esters), mometasone (e.g. as furoate), beclomethasone (e.g. as 17-propionate or 17,21-dipropionate esters), ciclesonide, triamcinolone (e.g. as acetonide), flunisolide, rofleponide or butixocort (e.g. as propionate ester)), e.g. for the treatment of a respiratory disorder (such as asthma or chronic obstructive pulmonary disease (COPD)), e.g. as described in WO 2007/008155 and/or WO 2007/008157.

A further therapeutic agent may include a 3-hydroxy-3-methylglutaryl coenzyme A (HMG CoA) reductase inhibitor (e.g. atorvastatin, fluvastatin, lovastatin, pravastatin, rosuvastatin, or simvastatin) (e.g. for oral administration), e.g. for the treatment of a cardiovascular disorder (such as atherosclerosis), e.g. as described in WO 2006/083214.

A further therapeutic agent may in particular include a non-steroid anti-inflammatory drug (NSAID; e.g. ibuprofen, naproxen, aspirin, celecoxib, diclofenac, etodolac, fenoprofen, indomethacin, ketoprofen, ketoralac, oxaprozin, nabumetone, sulindac, tolmetin, rofecoxib, valdecoxib, lumaricoxib, meloxicam, etoricoxib or parecoxib; or e.g. paracetamol, loxoprofen or aceclofenac; in particular celecoxib, paracetamol, ibuprofen or diclofenac) (e.g. for oral administration), e.g. for the treatment of an inflammatory disease or disorder (such as rheumatoid arthritis or osteoarthritis, and/or inflammatory pain), e.g. as described in WO 2005/025571. Celecoxib (a COX-2 inhibitor) can for example be administered orally at a dosage regimen of 100 mg or 200 mg (measured as the free base) once or twice daily.

A further therapeutic agent may in particular include a tumour necrosis factor α (TNFα) inhibitor (e.g. etanercept or an anti-TNFα antibody such as infliximab or adalimumab) (e.g. for parenteral administration such as subcutaneous or intravenous administration), e.g. for the treatment of an inflammatory disease or disorder (such as rheumatoid arthritis or osteoarthritis), e.g. as described in WO 2004/105798.

A further therapeutic agent may in particular include an anti-CD20 monoclonal antibody (e.g. for parenteral such as intravenous administration), such as ofatumumab (HuMax-CD20™, developed in part by Genmab AS) (e.g. ofatumumab for intravenous administration), rituximab, PRO70769, AME-133 (Applied Molecular Evolution), or hA20 (Immunomedics, Inc.); in particular ofatumumab or rituximab. This further therapeutic agent can e.g. be for the treatment of an inflammatory disease or disorder (such as rheumatoid arthritis or osteoarthritis, and/or inflammatory pain).

A further therapeutic agent may include 2-hydroxy-5-[[4-[(2-pyridinylamino) sulfonyl]phenyl]azo]benzoic acid (sulfasalazine), e.g. for the treatment of an inflammatory disease or disorder (such as rheumatoid arthritis or osteoarthritis; in particular rheumatoid arthritis), e.g. as described in WO 2004/105797.

A further therapeutic agent may in particular include N-[4-[[(2,4-diamino-6-pteridinyl)methyl]methylamino]benzoyl]-L-glutamic acid (methotrexate), e.g. for oral administration and/or e.g. for the treatment of an inflammatory disease or disorder (such as rheumatoid arthritis or osteoarthritis; in particular rheumatoid arthritis), e.g. as described in WO 2004/105796. For the treatment of rheumatoid arthritis, methotrexate can be administered to the human at a dosage regimen of 7.5 mg orally once weekly, or using divided oral doses of 2.5 mg at 12 hour intervals for 3 doses (7.5 mg total) as a course once weekly; the schedule can optionally be adjusted gradually to achieve an optimal response, but typically does not exceed a total weekly oral dose of 20 mg of methotrexate; once a response has been achieved, the methotrexate dose is typically reduced to the lowest possible effective dose.

A further therapeutic agent may include an inhibitor of pro TNFα convertase enzyme (TACE), e.g. for the treatment of an inflammatory disease or disorder (such as rheumatoid arthritis or osteoarthritis; in particular rheumatoid arthritis), e.g. as described in WO 2004/073704.

A further therapeutic agent may include:
a) sulfasalazine;
b) a statin (e.g. for oral administration), such as atorvastatin, lovastatin, pravastatin, simvastatin, fluvastatin, cerivastatin, crilvastatin, dalvastatin, rosuvastatin, tenivastatin, fluindostatin, velostatin, dalvastatin, nisvastatin, bervastatin, pitavastatin, rivastatin, glenvastatin, eptastatin, tenivastatin, flurastatin, rosuvastatin or itavastatin;
c) a glucocorticoid agent (e.g. for oral or skin-topical administration), such as dexamethasone, methylprednisolone, prednisolone, prednisone and hydrocortisone;
d) an inhibitor of p38 kinase (e.g. for oral administration);
e) an anti-IL-6-receptor antibody, e.g. an anti-IL-6-receptor monoclonal antibody (e.g. for parenteral such as intravenous administration);
f) anakinra;
g) an anti-IL-1 (e.g. IL-1β3) monoclonal antibody (e.g. for parenteral such as intravenous administration);
h) an inhibitor of JAK3 protein tyrosine kinase;
i) an anti-macrophage colony stimulation factor (M-CSF) monoclonal antibody; or
j) an anti-CD₂O monoclonal antibody (e.g. for parenteral such as intravenous administration), such as rituximab, ofatumumab (HuMax-CD20™, developed in part by Genmab AS) (e.g. ofatumumab for intravenous administration), PRO70769, AME-133 (Applied Molecular Evolution), or hA20 (Immunomedics, Inc.); in particular rituximab or ofatumumab;
   e.g. for the treatment of an IL-1 (e.g. IL-1β) mediated disease (such as rheumatoid arthritis or osteoarthritis, and/or inflammatory or neuropathic pain; in particular rheumatoid arthritis), e.g. as described in WO 2006/003517.

In particular, the further therapeutic agent or agents can be a therapeutic agent or agents capable of treating inflammatory pain, such as paracetamol and/or an opioid (such as morphine, fentanyl, oxycodone, tramadol, hydrocodone, hydromorphone, oxymorphone, methadone or buprenorphine; in particular morphine, fentanyl, oxycodone, or tramadol). This/these therapeutic agent(s), and/or the combination comprising this/these therapeutic agent(s), can be for the treatment of inflammatory pain, e.g. in a mammal such as a human. For example, paracetamol can be administered at a human oral dosage regimen of 500 mg to 1000 mg (e.g. 500 mg, 650 mg or 1000 mg, in particular 650 mg) of paracetamol (measured as the free base/free compound), administered two, three or four times daily.

In a particular embodiment of the invention, the further therapeutic agent or agents can be a therapeutic agent or agents capable of treating neuropathic pain, such as:
an opioid (such as morphine, fentanyl, oxycodone, tramadol, hydrocodone, hydromorphone, oxymorphone, methadone or buprenorphine; in particular morphine, fentanyl, oxycodone, or tramadol, most particularly morphine),
a monoamine reuptake inhibitor (such as duloxetine or amytriptyline),
pregabalin,
gabapentin,
gabapentin enacarbil (XP13512), and/or
carbamazepine.

This/these therapeutic agent(s), and/or the combination comprising this/these therapeutic agent(s), can be for the treatment of neuropathic pain, e.g. in a mammal such as a human.

For example, pregabalin can be administered orally e.g. for neuropathic pain; e.g. at a human oral dosage regimen of 150 mg to 600 mg total pregabalin per day (measured as the free base), split between two to three doses per day. For example, for postherpetic neuralgia (a neuropathic pain condition), pregabalin can be administered at a starting oral dosage regimen of 150 mg total pregabalin per day (split between 2 or 3 doses per day), escalating (e.g. in about one week) to an oral dosage regimen of 300 mg pregabalin total per day, and optionally escalating up to a maximum oral dosage regimen of 600 mg total pregabalin per day. For painful diabetic neuropathy (another neuropathic pain condition), an oral dosage regimen of 150 mg to 300 mg total pregabalin per day can be administered. For fibromyalgia, an oral dosage regimen of 150 mg to 450 mg (e.g. 300 or 450 mg) total pregabalin per day can be administered. Pregabalin can e.g. be administered separately from the compound of formula (1) or the salt thereof.

For example, gabapentin can be administered orally, e.g. for neuropathic pain. Oral dosage units can e.g. contain 100 mg, 300 mg, 400 mg, 600 mg or 800 mg of gabapentin (measured as the free base/acid). The gabapentin dosage regimen for neuropathic pain can e.g. be from 300 mg once, twice or three times per day up to a total dose of 3600 mg/day. Some gradual up-titration of the dosage regimen is usually performed. For example, for peripheral neuropathic pain in adults, gabapentin therapy can be initiated by titrating the dose thus: day 1=300 mg of gabapentin (measured as the free base/acid) once a day, day 2=300 mg two times a day, and day 3=300 mg three times a day; alternatively the starting dose can be 900 mg/day of gabapentin (measured as the free base/acid), administered as three equally divided doses. Thereafter, e.g. based on individual patient response and tolerability, the dose can be further increased, typically in 300 mg/day increments every 2-3 days, up to a maximum total dose of 3600 mg/day of gabapentin (measured as the free base/acid). Slower titration of gabapentin dosage may be appropriate for individual patients. The minimum time to reach a total dose of 1800 mg/day is typically one week, to reach 2400 mg/day is typically a total of 2 weeks, and to reach 3600 mg/day is typically a total of 3 weeks. Gabapentin can e.g. be administered separately from the compound of formula (1) or the salt thereof.

For example, gabapentin enacarbil (XP13512, (±)-1-([(α-isobutanoyloxyethoxy)carbonyl]-aminomethyl)-1-cyclohexane acetic acid, which is a prodrug of gabapentin) can be administered orally, e.g. to a human, e.g. separately from the compound of formula (1) or the salt thereof. In one embodiment, gabapentin enacarbil (XP13512) is for example administered orally, e.g. to a human such as a human adult, e.g. at a total daily dose having an equivalent molar quantity of gabapentin enacarbil as the molar quantity present in 900 mg/day to 3600 mg/day of gabapentin (see e.g. page 81 lines 24-32 of WO 02/100347). A 600 mg dose of gabapentin enacarbil (measured as the free acid) contains the molar equivalent of 312 mg of gabapentin. See also K. C. Cundy et al., “Clinical Pharmacokinetics of XP13512, a Novel Transported Prodrug of Gabapentin”, J. Clin. Pharmacol., 2008, e-publication 30 Sep. 2008, incorporated herein by reference, and the Materials and Methods—Formulation and Study Designs sections therein, for examples of some oral doses, dosage regimens and formulations of XP13512 used in human pharmacokinetic studies.

In a particular embodiment of the invention, when the further therapeutic agent includes an opioid (such as morphine, fentanyl, oxycodone, tramadol, hydrocodone, hydromorphone, oxymorphone, methadone or buprenorphine; in particular morphine, fentanyl, oxycodone, or tramadol), then the opioid and/or the combination comprising the opioid is for the treatment of pain, in particular inflammatory or neuropathic pain, e.g. in a mammal such as a human.

When the compounds are used in combination with other therapeutic agents, the compounds may be administered either sequentially or simultaneously by any convenient route.

The invention thus provides, in a further aspect, a combination comprising a compound of formula (1) or a pharmaceutically acceptable salt thereof together with a further therapeutic agent or agents (e.g. as defined herein).

The individual components of the combination of the invention (i.e. the compound of formula (1) or the salt thereof, and the further therapeutic agent or agents) may be present as separate pharmaceutical formulations/compositions, or may be present as a combined pharmaceutical formulation/composition (e.g. may be together in a single combined oral dosage form, e.g. a single combined tablet or capsule). The individual components of this combination can for example be administered either sequentially in separate pharmaceutical formulations/compositions (e.g. oral), or simultaneously in separate or combined pharmaceutical formulation(s)/composition(s) (e.g. oral); in a particular embodiment they are administered sequentially in separate pharmaceutical formulations/compositions (e.g. oral).

The combinations referred to herein may optionally be presented for use in the form of a pharmaceutical formulation and thus pharmaceutical formulations comprising a combination as defined herein together with a pharmaceutically acceptable carrier or excipient comprise a further aspect of the invention. The individual components of such combinations may be administered either sequentially or simultaneously in separate or combined pharmaceutical formulations.

When a compound of formula (1) or a pharmaceutically acceptable salt thereof is used in combination with a second therapeutic agent active against the same disease state the dose of each compound may differ from that when the compound is used alone.

The following descriptions and Examples illustrate the compounds of the invention and methods for their preparation but are not intended to be limiting.

### EXAMPLES

Abbreviations, some of which may be used herein, include the following:
HEPES 4-(2-hydroxyethyl)-1-piperazine-1-ethanesulfonic acid

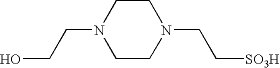

eq equivalents
HPLC high performance liquid chromatography
h hours
min minutes
LCMS or LC/MS liquid chromatography/mass spectrometry
MS mass spectrometry
RT room temperature (ambient temperature); this is usually in the range of about 18 to about 25° C., or a sub-range within this range, unless otherwise disclosed herein.

The general methods (a)-(c), along with the synthetic methods outlined in Schemes 1 to 3 above, for the preparation of compounds of the present invention are further illustrated by the following examples.

### Example 1

N-{[4-fluoro-2-(trifluoromethyl)phenyl]methyl}-2-(2-methylpropyl)-3-isothiazolidinecarboxamide 1,1-dioxide (E1) (in a form prepared or obtainable from L-homocystine)

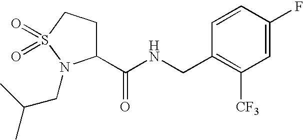

2-(2-methylpropyl)-3-isothiazolidinecarboxylic acid 1,1-dioxide (0.080 g, 0.36 mmol, prepared as described below) was dissolved in dimethylformamide (8 ml) and to this was added 1-[4-fluoro-2-(trifluoromethyl)phenyl]methanamine (0.139 g, 0.72 mmol), then diisopropylethylamine (0.140 g, 1.08 mmol), 1-hydroxybenzotriazole (0.122 g, 0.90 mmol), and N-(3-dimethylaminopropyl)-N′-ethylcarbodiimide hydrochloride (0.173 g, 0.90 mmol). The mixture was stirred at room temperature for 24 hrs then diluted with water and extracted with ethyl acetate. The combined organic layers were then washed with saturated aqueous sodium chloride solution and then with water, then dried with anhydrous sodium sulphate and evaporated. The resulting residue was purified by preparative HPLC to give pure N-{[4-fluoro-2-(trifluoromethyl)phenyl]methyl}-2-(2-methylpropyl)-3-isothiazolidinecarboxamide 1,1-dioxide (0.045 g).
MS [M+H]⁺=397.07, HPLC^{a} retention time=3.35 minutes.

The 2-(2-methylpropyl)-3-isothiazolidinecarboxylic acid 1,1-dioxide used in the above procedure was prepared as follows:
(i) Ethyl (3S)-3-isothiazolidinecarboxylate 1,1-dioxide was prepared from L-homocystine in a manner analogous to that described in Luisi, Grazia; Pinnen, Francesco. Archie der Pharmazie (Weinheim, Germany) (1993), 326(3), 139-41.
(ii) Ethyl (3S)-3-isothiazolidinecarboxylate 1,1-dioxide (0.100 g, 0.52 mmol) was dissolved in dimethylformamide (2 ml) and treated with potassium carbonate (0.286 g, 2.07 mmol) and then with isobutyl bromide (0.142 g, 1.04 mmol). The mixture was stirred at room temperature for 4.5 hrs and then heated at 70° C. for 2 hrs. The mixture was then diluted with water and the resulting mixture was extracted with ethyl acetate (3 times). The combined organic fractions were washed three times with water, then dried over anhydrous sodium sulphate and evaporated to give a residue which was purified further by silica-gel column chromatography, eluting with a 10:1 mixture of petroleum ether and ethyl acetate respectively, to give pure ethyl 2-(2-methylpropyl)-3-isothiazolidinecarboxylate 1,1-dioxide (0.100 g).
(iii) Ethyl 2-(2-methylpropyl)-3-isothiazolidinecarboxylate 1,1-dioxide (0.400 g, 1.6 mmol) was dissolved in tetrahydrofuran (4 ml) and treated with lithium hydroxide monohydrate (0.270 g, 6.4 mmol). The mixture was stirred at room temperature for 4 hrs and then poured into water. The aqueous mixture was extracted with ethyl acetate and then the aqueous layer was treated with an aqueous HCl solution (10%) at 0° C. and then extracted with ethyl acetate. The combined organic layer was dried over Na₂SO₄ and concentrated to give 2-(2-methylpropyl)-3-isothiazolidinecarboxylic acid 1,1-dioxide (0.320 g).

### Examples 2-32

### In Forms Prepared or Obtainable from L-Homocystine)

In a manner analogous to that described for Example 1 above the compounds tabulated below (Table 1, in forms prepared or obtainable from L-homocystine) were prepared by substituting the appropriate amine and/or alkyl halide, all of which are available from commercial sources or can be prepared using routes described previously in the chemical literature, for the 1-[4-fluoro-2-(trifluoromethyl)phenyl]methanamine and/or isobutyl bromide respectively that were used in the above procedure.

TABLE 1

| Ex- | | | Retention |
|---|---|---|---|
| ample | | [M + H]⁺ | time* |
| no. | Chemical name | [2M + Na]⁺ | (mins) |
| E2 | 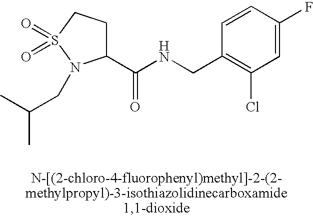 | — 746.82 | 3.15^{a} |
| E3 | 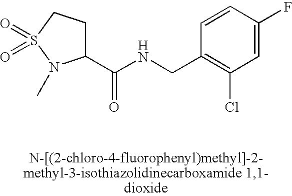 | 320.95 662.71 | 1.80^{b} |
| E4 | 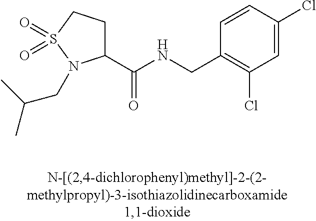 | 379.04 778.75 | 3.41^{a} |
| E5 | 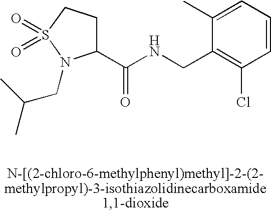 | 359.03 738.86 | 3.27^{a} |
| E6 | 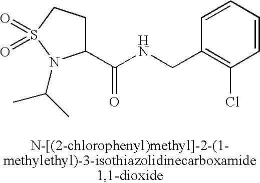 | 331.0 682.6 | 2.15^{b} |
| E7 | 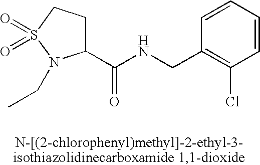 | — 654.75 | 2.99^{c} |
| E8 | 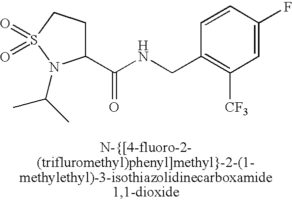 | 382.99 786.65 | 2.50^{b} |
| E9 | 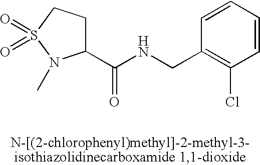 | 303.2 — | 1.86^{b} |
| E10 | 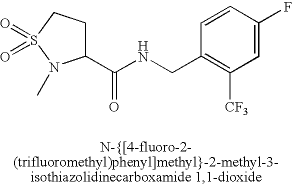 | 730.67 | 2.03^{b} |
| E11 | 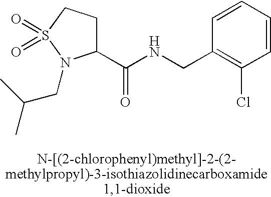 | 345.0 710.6 | 2.50^{b} |
| E12 | 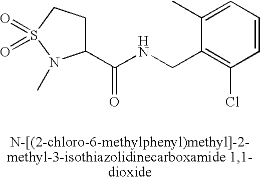 | — 654.73 | 2.20^{d} |
| E13 | 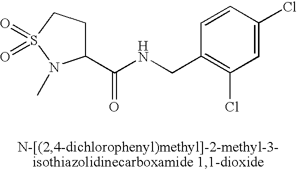 | — 694.56 | 2.28^{b} |
| E14 | 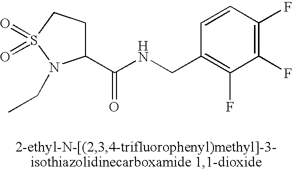 | 337.0 — | 1.99^{d} |
| E15 | 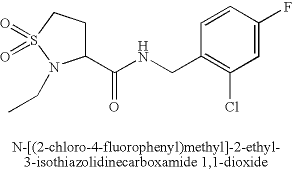 | 334.9 — | 2.15^{d} |
| E16 | 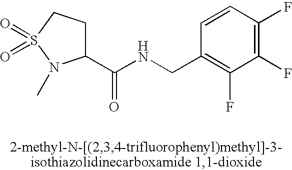 | — 666.65 | 2.82^{c} |
| E17 | 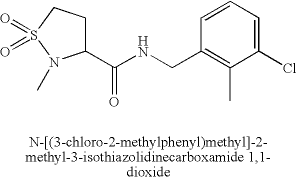 | 316.9 — | 2.31^{d} |
| E18 | 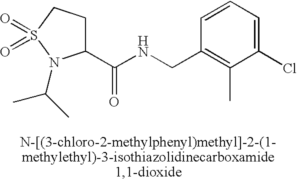 | — 710.75 | 2.63^{d} |
| E19 | 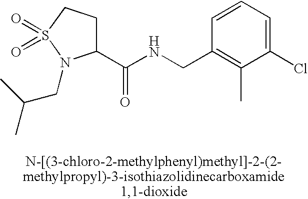 | 359.0 — | 2.81^{b} |
| E20 | 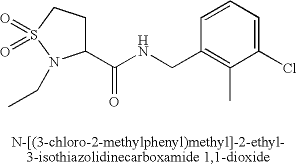 | 330.9 — | 3.32^{c} |
| E21 | 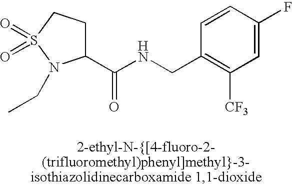 | 369.0 — | 3.39^{c} |
| E22 | 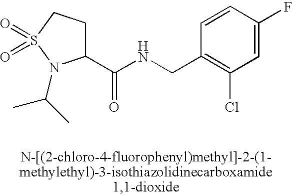 | 349.0 — | 2.41^{d} |
| E23 | 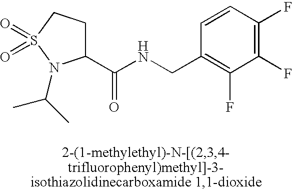 | 350.9 — | 2.26^{d} |
| E24 | 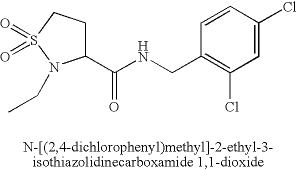 | 350.9 — | 3.41^{c} |
| E25 | 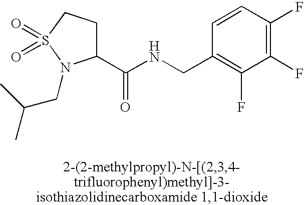 | 365.0 — | 2.49^{b} |
| E26 | 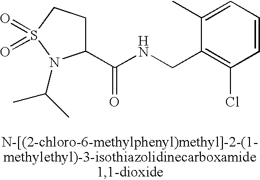 | 344.9 — | 2.54^{d} |
| E27 | 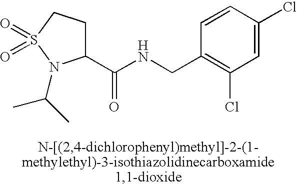 | 364.9 — | 2.73^{d} |
| E28 | 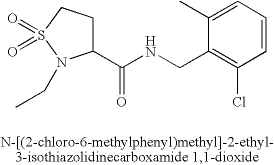 | 331.0 — | 2.36^{d} |
| E29 | 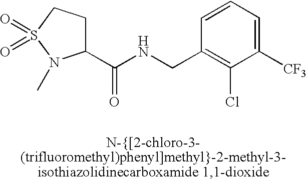 | 371 — | 2.44^{f} |
| E30 | 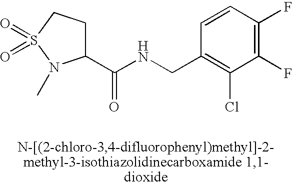 | 339 — | 2.23^{f} |
| E31 | 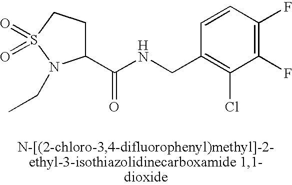 | 353 — | 2.41^{f} |
| E32 | 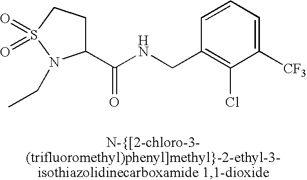 | 385 — | 2.61^{f} |

*superscript denotes HPLC method as detailed below

### Example 33

2-ethyl-N-{[4-fluoro-2-(trifluoromethyl)phenyl]methyl}tetrahydro-2H-1,2-thiazine-3-carboxamide 1,1-dioxide (E33) (in a form prepared or obtainable from 1,1-dimethylethyl N-{[(1,1-dimethylethyl)oxy]carbonyl}-5-hydroxy-L-norvalinate)

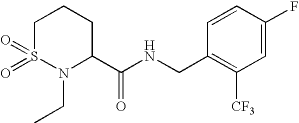

2-ethyl-N-{[4-fluoro-2-(trifluoromethyl)phenyl]methyl}tetrahydro-2H-1,2-thiazine-3-carboxamide 1,1-dioxide was prepared in an analogous manner to that described for N-{[4-fluoro-2-(trifluoromethyl)phenyl]methyl}-2-(2-methylpropyl)-3-isothiazolidinecarboxamide 1,1-dioxide (E1) above but using 2-ethyltetrahydro-2H-1,2-thiazine-3-carboxylic acid 1,1-dioxide in the place of 2-(2-methylpropyl)-3-isothiazolidinecarboxylic acid 1,1-dioxide.
MS [M+H]⁺=383.05, HPLC^{e} retention time=3.89 minutes.

The 2-ethyltetrahydro-2H-1,2-thiazine-3-carboxylic acid 1,1-dioxide used in the above procedure was prepared from 1,1-dimethylethyl N-{[(1,1-dimethylethyl)oxy]carbonyl}-5-hydroxy-L-norvalinate in a manner analogous to that described in R. J. Chemey, et. al., J. Med. Chem., 2004, 47, 2981-2983 (also see WO2002028846) but using ethyl iodide in place of the benzylic halides described.

### Examples 34-53

### In Forms Prepared or Obtainable from 1,1-dimethylethyl N-{[(1,1-dimethylethyl)oxy]carbonyl}-5-hydroxy-L-norvalinate

In a manner analogous to that described for Example 33 above the compounds tabulated below (Table 2, in forms prepared or obtainable from 1,1-dimethylethyl N-{[(1,1-dimethylethyl)oxy]carbonyl}-5-hydroxy-L-norvalinate) were prepared by substituting the appropriate amine and/or alkyl halide, all of which are available from commercial sources or can be prepared using routes described previously in the chemical literature, for the 1-[4-fluoro-2-(trifluoromethyl)phenyl]methanamine and/or ethyl iodide respectively that were used in the above procedure.

TABLE 2

| | | | Retention |
|---|---|---|---|
| | | [M + H]⁺ | time* |
| Example no. | Chemical name | [2M + Na]⁺ | (mins) |
| E34 | 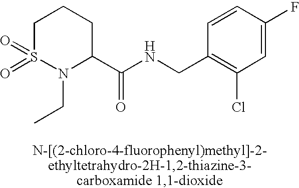 | 349.01 — | 3.71^{e} |
| E35 | 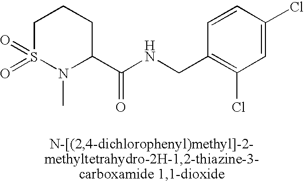 | 351.0 724.6 | 3.68^{e} |
| E36 | 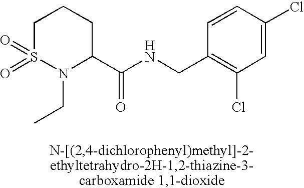 | 365.0 — | 2.87^{b} |
| E37 | 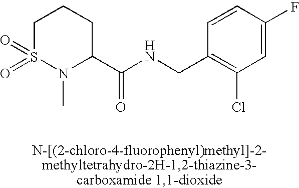 | 334.98 690.66 | 2.29^{d} |
| E38 | 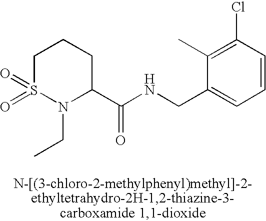 | 345.05 710.74 | 3.11^{a} |
| E39 | 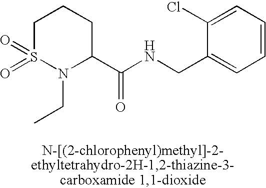 | 331.06 682.78 | 2.29^{b} |
| E40 | 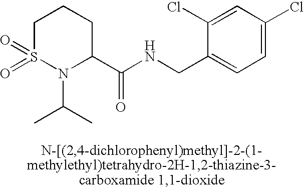 | — 780.71 | 3.12^{d} |
| E41 | 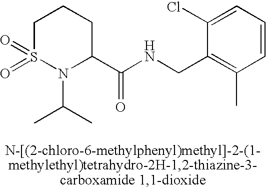 | 358.99 738.78 | 2.78^{b} |
| E42 | 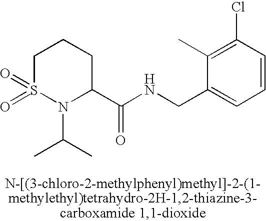 | 358.97 738.77 | 2.86^{b} |
| E43 | 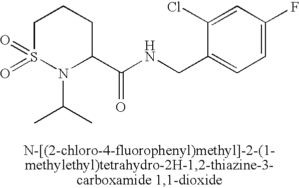 | 362.96 746.75 | 2.65^{b} |
| E44 | 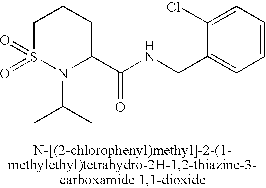 | 344.98 710.77 | 2.54^{b} |
| E45 | 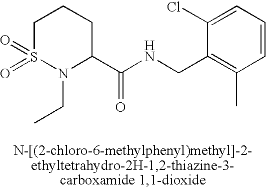 | 345.05 710.78 | 2.58^{b} |
| E46 | 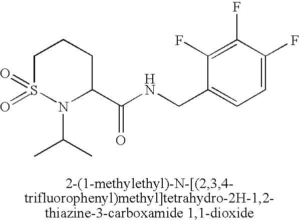 | 364.98 750.72 | 2.54^{b} |
| E47 | 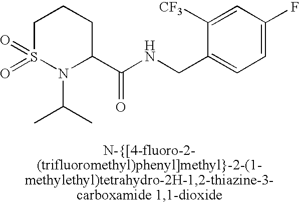 | 397.01 814.73 | 2.84^{b} |
| E48 | 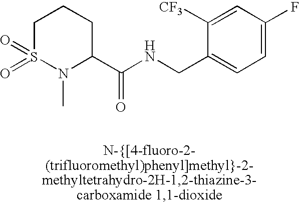 | 369.0 758.6 | 2.47^{b} |
| E49 | 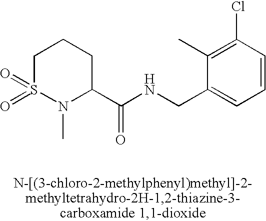 | 331.03 682.74 | 2.51^{b} |
| E50 | 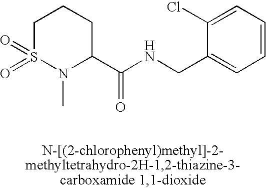 | 317.0 654.7 | 2.02^{b} |
| E51 | 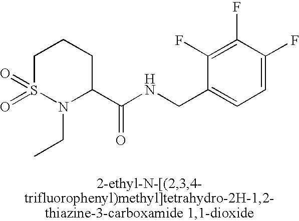 | 351.0 722.7 | 2.38^{b} |
| E52 | 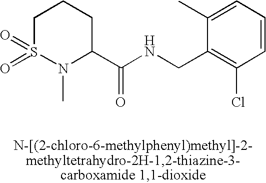 | 331.0 682.7 | 2.39^{b} |
| E53 | 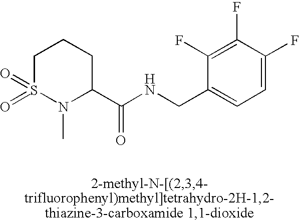 | 337.0 694.6 | 2.05^{b} |

*superscript denotes HPLC method as detailed below

### Mass-Directed Automated HPLC

Where applicable, purification by mass-directed automated HPLC was carried out using the following apparatus and conditions:

### Hardware

Waters 2525 Binary Gradient Module
Waters 515 Makeup Pump
Waters Pump Control Module
Waters 2767 Inject Collect
Waters Column Fluidics Manager
Waters 2996 Photodiode Array Detector
Waters ZQ Mass Spectrometer
Gilson 202 fraction collector
Gilson Aspec waste collector

### Software

Waters MassLynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid
B: Organic solvent=Acetonitrile+0.1% Formic Acid
Make up solvent=Methanol: Water 80:20
Needle rinse solvent=Methanol

### Methods

There are five methods used depending on the analytical retention time of the compound of interest. They have a 13.5-minute runtime, which comprises a 10-minute gradient followed by a 3.5 minute column flush and re-equilibration step.
Large/Small Scale 1.0-1.5=5-30% B
Large/Small Scale 1.5-2.2=15-55% B
Large/Small Scale 2.2-2.9=30-85% B
Large/Small Scale 2.9-3.6=50-99% B
Large/Small Scale 3.6-5.0=80-99% B (in 6 minutes followed by 7.5 minutes flush and re-equilibration)

### Flow Rate

All of the above methods have a flow rate of either 20 mls/min (Small Scale) or 40 mls/min (Large Scale).

### Liquid Chromatography/Mass Spectrometry

Analysis of the above Examples by Liquid Chromatography/Mass Spectrometry (LC/MS) was carried out using the apparatus and conditions indicated in the methods shown below:

### Liquid Chromatography:

Method (a): (Examples 1-2, 4-5, 38)

### Hardware

Shimadzu LC-20AB Pump
Shimadzu SIL-20AB Auto sampler
Shimadzu SPD-M20A DAD Detector
Shimadzu CTO-20AC Oven
Shimadzu DGU-20A3 Degasser

### Software

Shimadzu CLASS-VP

### Column

YMC HPLC COLUMN: 50*4.6 mm I.D., S-5 μm, 12 nm

### Solvents

A: 4 L Water+2.75 mL TFA
B: 4 L Acetonitrile+2.5 mL TFA
The generic method used has a 6 minute runtime.

| Time/min | % B |
|---|---|
| 0.01 | 10 |
| 4.00 | 80 |
| 6.00 | 80 |
| 6.01 | 10 |
| 7.80 | 10 |

The above method has a flow rate of 3 ml/mins.
The injection volume for the generic method is 5ul.
The column temperature is 40 deg.
The UV detection range is from 190 to 370 nm.

### Method (b): (Examples 3, 6, 8-11, 13, 19, 25, 36, 39, 41-52, 53) Hardware

Shimadzu LC-20AB Pump
Shimadzu SIL-20AB Auto sampler
Shimadzu SPD-M20A DAD Detector
Shimadzu CTO-20AC Oven
Shimadzu DGU-20A3 Degasser

### Software

Shimadzu CLASS-VP

### Column

YMC HPLC COLUMN: 50*4.6 mm I.D., S-5 μm, 12 nm

### Solvents

A: 4 L Water+2.75 mL TFA
B: 4 L Acetonitrile+2.5 mL TFA
The generic method used has a 6 minute runtime.

| Time/min | % B |
|---|---|
| 0.01 | 10 |
| 4.00 | 80 |
| 6.00 | 80 |
| 6.01 | 10 |
| 7.80 | 10 |

The above method has a flow rate of 3 ml/mins.
The injection volume for the generic method is 5 ul.
The column temperature is 40 deg.
The UV detection range is from 190 to 370 nm.

### Method (c): (Examples 7, 16, 20-21, 24) Hardware

Shimadzu LC-20AB Pump
Shimadzu SIL-20AB Auto sampler
Shimadzu SPD-M20A DAD Detector
Shimadzu CTO-20AC Oven
Shimadzu DGU-20A3 Degasser

### Software

Shimadzu CLASS-VP

### Column

XB-C18, 5 μm, 4.6*50 mm

### Solvents

A: 1 L Water+0.5 mL NH₃H2O
B: Acetonitrile
The generic method used has a 6 minute runtime.

| Time/min | % B |
|---|---|
| 0.01 | 10 |
| 4.00 | 80 |
| 6.00 | 80 |
| 6.01 | 10 |
| 7.80 | 10 |

The above method has a flow rate of 3 ml/mins.
The injection volume for the generic method is 5 ul.
The column temperature is 40 deg.
The UV detection range is from 190 to 370 nm.

### Method (d): (Examples 12, 14-15, 17-18, 22-23, 26-28, 37, 40) Hardware

Shimadzu LC-20AB Pump
Shimadzu SIL-20AB Auto sampler
Shimadzu SPD-M20A DAD Detector
Shimadzu CTO-20AC Oven
Shimadzu DGU-20A3 Degasser

### Software

Shimadzu CLASS-VP

### Column

YMC HPLC COLUMN: 50*4.6 mm I.D., S-5 μm, 12 nm

### Solvents

A: 4 L Water+2.75 mL TFA
B: 4 L Acetonitrile+2.5 mL TFA
The generic method used has a 6 minute runtime.

| Time/min | % B |
|---|---|
| 0.01 | 10 |
| 4.00 | 80 |
| 6.00 | 80 |
| 6.01 | 10 |
| 7.80 | 10 |

The above method has a flow rate of 3 ml/mins.
The injection volume for the generic method is 5 ul.
The column temperature is 40 deg.
The UV detection range is from 190 to 370 nm.
Method (e): (Examples 33-35)

### Hardware

Shimadzu LC-20AB Pump
Shimadzu SIL-20AB Auto sampler
Shimadzu SPD-M20A DAD Detector
Shimadzu CTO-20AC Oven
Shimadzu DGU-20A3 Degasser

### Software

Shimadzu CLASS-VP

### Column

YMC HPLC COLUMN: 50*4.6 mm I.D., S-5 μm, 12 nm

### Solvents

A: 4 L Water+2.75 mL TFA
B: 4 L Acetonitrile+2.5 mL TFA
The generic method used has a 6 minute runtime.

| Time/min | % B |
|---|---|
| 0.01 | 10 |
| 4.00 | 80 |
| 6.00 | 80 |
| 6.01 | 10 |
| 7.80 | 10 |

The above method has a flow rate of 3 ml/mins.
The injection volume for the generic method is 5ul.
The column temperature is 40 deg.
The UV detection range is from 190 to 370 nm.
Mass Spectrometry for methods (a)-(e) above:
Shimadzu LC-2010A HT AB API 150EX or Shimadzu LC-2010A HT Thermo LCQ Advantage.

### Method (f) (Examples 29-32) Hardware

Agilent 1100 Gradient Pump
Agilent 1100 Autosampler
Agilent 1100 DAD Detector
Agilent 1100 Degasser
Agilent 1100 Oven
Agilent 1100 Controller
Waters ZQ Mass Spectrometer
Sedere Sedex 85

### Software

Waters MassLynx version 4.0 SP2

### Column

The column used is a Waters Atlantis, the dimensions of which are 4.6 mm×50 mm. The stationary phase particle size is 3 μm.

### Solvents

A: Aqueous solvent=Water+0.05% Formic Acid
B: Organic solvent=Acetonitrile+0.05% Formic Acid
The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

The above method has a flow rate of 3 ml/mins.
The injection volume for the generic method is 5 ul.
The column temperature is 30 deg.
The UV detection range is from 220 to 330 nm.

### Pharmacological Data

Compounds or salts of the invention may be tested for in vitro biological activity at the P2X7 receptor in accordance with the following studies:

### Eithidium Accumulation Assay

Studies were performed using NaCl assay buffer of the following composition: 140 mM NaCl, 10 mM HEPES [4-(2-hydroxyethyl)-1-piperazine-1-ethanesulfonic acid], 5 mM N-methyl-D-glucamine, 5.6 mM KCl, 10 mM D-glucose, 0.5 mM CaCl₂(pH 7.4).

Human Embryonic Kidney (HEK) 293 cells, stably expressing human recombinant P2X7 receptors, were grown in poly-D-lysine pretreated 96 well plates for 18-24 hours. (The cloning of the human P2X7 receptor is described in U.S. Pat. No. 6,133,434, e.g. see Example 3 therein). The cells were washed twice with 350 μl of the assay buffer, before addition of 50 μl of the assay buffer containing the putative P2X7 receptor antagonist compound. (A small amount of dimethyl sulfoxide, for initially dissolving the compound, is optionally used and present in this 50 μl test compound sample.) The cells were then incubated at room temperature (19-21° C.) for 30 min before addition of ATP and ethidium (100 μM final assay concentration). The ATP concentration was chosen to be close to the EC₈₀ for the receptor type and was 1 mM for studies on the human P2X7 receptor. Incubations were continued for 8 or 16 min and were terminated by addition of 25 μl of 1.3M sucrose containing 4 mM of the P2X7 receptor antagonist Reactive Black 5 (Aldrich). Cellular accumulation of ethidium was determined by measuring fluorescence (excitation wavelength of 530 nm and emission wavelength of 620 nm) from below the plate with a Can berra Packard Fluorocount (14 Station Road, Pangbourne, Reading, Berkshire RG8 7AN, United Kingdom) or a FlexStation II 384 from Molecular Molecular Devices (660-665 Eskdale Road, Wokingham, Berkshire RG41 5TS, United Kingdom). Antagonist plC₅₀ values for blocking ATP responses were determined using iterative curve fitting techniques.

### Fluorescent Imaging Plate Reader (FLIPR) Ca Assay

Studies were performed using NaCl assay buffer of the following composition for human P2X7: 137 mM NaCl; 20 mM HEPES [4-(2-hydroxyethyl)-1-piperazine-1-ethanesulfonic acid]; 5.37 mM KCl; 4.17 mM NaHCO₃; 1 mM CaCl₂; 0.5 mM MgSO₄; and 1 g/L of D-glucose (pH 7.4).

Human Embryonic Kidney (HEK) 293 cells, stably expressing human recombinant P2X7 receptors, were grown in poly-D-lysine pretreated 384 well plates for 24 hours at room temperature (for a time sufficient for growth of a homogeneous layer of cells at the bottom of the wells). Alternatively, human osteosarcoma (U-2OS) cells (commercially available), transduced with modified Baculovirus (BacMam) vector to deliver the gene coding for human P2X7 receptor (i.e. transiently expressing human recombinant P2X7 receptors), were grown in substantially the same conditions as for the HEK293 cells except that the well plates were not pre-treated with poly-D-lysine. (The cloning of the human P2X7 receptor is described in U.S. Pat. No. 6,133,434, e.g. see Example 3 therein). The cells were washed three times with 80 μl of assay buffer, loaded for 1 h at 37° C. with 2 μM Fluo-4-AM [4-(6-acetoxymethoxy-2,7-difluoro-3-oxo-9-xanthenyl)-4′-methyl-2,2′-(ethylenedioxy)dianiline-N,N,N′,N′-tetraacetic acid tetrakis(acetoxymethyl) ester], a Ca²⁺-sensitive, cell-permeable, fluorescent dye (Tef Labs. Inc., 9415 Capitol View Drive, Austin, Tex. 78747, USA), washed three times again (3×80 μl), and left with 30 μl buffer before the addition of 10 μl of the assay buffer containing the putative P2X7 receptor antagonist compound, the compound being added at 4× the final assay concentration chosen. The solution of the putative P2X7 receptor antagonist compound was created by (i) dissolving the compound in dimethyl sulfoxide (DMSO) to create a stock solution in DMSO at 200× the final assay concentration, and (ii) mixing 1 μl of the stock solution of the compound in DMSO with 50 μl of the assay buffer to create a solution at about 4× the final assay concentration. The cells were then incubated at room temperature for 30 mins before addition (online, by FLIPR384 or FLIPR3 instrument (Molecular Devices, 1311 Orleans Drive, Sunnyvale, Calif. 94089-1136, USA)) of 10 μlof the assay buffer containing benzoylbenzoyl-ATP (BzATP) such as to create a 60 μM final assay concentration of BzATP (BzATP was added at 5× this final concentration). The BzATP concentration was chosen to be close to the EC₈₀ for the receptor type. Incubations and reading were continued for 90 sec, and intracellular calcium increase was determined by measuring fluorescence (excitation wavelength of 488 nm and emission wavelength of 516 nm) from below the plate, with FLIPR charged-coupled device (CCD) camera. Antagonist plC₅₀ values for blocking BzATP responses were determined using iterative curve fitting techniques.

The compounds of Examples 1 to 10, 12 to 32, 33 to 38, 40, 42, 45, 49, 50, 51, 52 and 53 were tested in the above FLIPR Ca Assay (or a slightly modified version thereof) and/or in the Ethidium Accumulation Assay (or a slightly modified version thereof) for human P2X7 receptor antagonist activity, and
were found to have pIC50 values of 4.7 or more in the FLIPR Ca Assay or a slightly modified version thereof, and/or
were found to have pIC50 values of from about 5.5 to about 8.0 (typically as a mean of more than one measurement) in the Ethidium Accumulation Assay or a slightly modified version thereof.

Examples 11, 39, 41, 43, 44, 46, 47 and 48 were found to have pIC50 values of from about 4.8 to about 5.4 (typically as a mean of more than one measurement) in the Ethidium Accumulation Assay or a slightly modified version thereof.

Examples 3, 4, 13, 15, 17, 18, 20, 21, 22, 24, 27, 29, 30, 31, 32, 35 and 36 were found to have pIC50 values of from about 6.5 to about 8.0 (typically as a mean of more than one measurement) in the Ethidium Accumulation Assay or a slightly modified version thereof.

1-18. (canceled)

19. A compound of formula (1) or a pharmaceutically acceptable salt thereof: 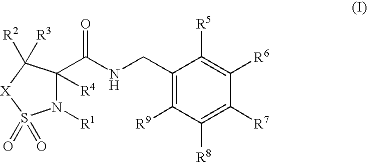
wherein:
R¹ represents C₁₋₄ alkyl optionally substituted with 1, 2, or 3 halogen atoms;
X represents —(CR¹⁰R¹¹)ₙ—;
R¹⁰ and R¹¹ independently represent hydrogen, halogen, or C₁₋₆ alkyl; wherein said C₁₋₆ alkyl group may be optionally substituted with 1, 2, or 3 halogen atoms;
n represents an integer being 1 or 2, such that when n represents 2, R¹ represents a group other than isobutyl;
R², R³, and R⁴ independently represent hydrogen, fluorine, or methyl; and
R⁵, R⁶, R⁷, R⁸, and R⁹ independently represent hydrogen, halogen, cyano, C₁₋₆ alkyl, C₂₋₆ alkenyl, C₂₋₆ alkynyl, C₃₋₆ cycloalkyl, wherein any of said C₁₋₆ alkyl, C₂₋₆ alkenyl, C₂₋₆ alkynyl or C₃₋₆ cycloalkyl groups may be optionally substituted with 1, 2, or 3 halogen atoms,
such that at least one of R⁵ and R⁹ represents a group other than hydrogen.

20. The compound or salt according to claim 19, wherein R¹ represents methyl, ethyl, or isopropyl.

21. The compound or salt according to claim 20, wherein R¹ represents methyl or ethyl.

22. The compound or salt according to claim 19, wherein n represents 1.

23. The compound or salt according to claim 19, wherein X represents —CH₂—.

24. The compound or salt according to claim 19, wherein R², R³, and R⁴ each represent hydrogen.

25. The compound or salt according to claim 19, wherein R⁵ represents hydrogen, halogen, or C₁₋₆ alkyl optionally substituted with 1, 2, or 3 halogen atoms.

26. The compound or salt according to claim 19, wherein R⁶ represents hydrogen or halogen.

27. The compound or salt according to claim 19, wherein R⁷ represents hydrogen or halogen.

28. The compound or salt according to claim 19, wherein R⁸ represents hydrogen, halogen, or C₁₋₆ alkyl optionally substituted with 1, 2, or 3 halogen atoms.

29. The compound or salt according to claim 19, wherein R⁹ represents hydrogen, halogen, or C₁₋₆ alkyl optionally substituted with 1, 2, or 3 halogen atoms.

30. The compound or salt according to claim 19, wherein:
R⁵ represents hydrogen, fluorine, chlorine, methyl, or —CF₃;
R⁶ represents hydrogen, fluorine, or chlorine;
R⁷ represents hydrogen, fluorine, or chlorine;
R⁸ represents hydrogen, fluorine, chlorine, or —CF₃; and
R⁹ represents hydrogen, fluorine, chlorine, methyl, or —CF₃;
such that at least one of R⁵ and R⁹ represents a group other than hydrogen.

31. The compound or salt according to claim 19 which is:
N-{[4-fluoro-2-(trifluoromethyl)phenyl]methyl}-2-(2-methylpropyl)-3-isothiazolidinecarboxamide 1,1-dioxide;
N-[(2-chloro-4-fluorophenyl)methyl]-2-(2-methylpropyl)-3-isothiazolidinecarboxamide 1,1-dioxide;
N-[(2-chloro-4-fluorophenyl)methyl]-2-methyl-3-isothiazolidinecarboxamide 1,1-dioxide;
N-[(2,4-dichlorophenyl)methyl]-2-(2-methylpropyl)-3-isothiazolidinecarboxamide 1,1-dioxide;
N-[(2-chloro-6-methylphenyl)methyl]-2-(2-methylpropyl)-3-isothiazolidinecarboxamide 1,1-dioxide;
N-[(2-chlorophenyl)methyl]-2-(1-methylethyl)-3-isothiazolidinecarboxamide 1,1-dioxide;
N-[(2-chlorophenyl)methyl]-2-ethyl-3-isothiazolidinecarboxamide 1,1-dioxide;
N-{[4-fluoro-2-(trifluoromethyl)phenyl]methyl}-2-(1-methylethyl)-3-isothiazolidinecarboxamide 1,1-dioxide;
N-[(2-chlorophenyOmethyl]-2-methyl-3-isothiazolidinecarboxamide 1,1-dioxide;
N-{[4-fluoro-2-(trifluoromethyl)phenyl]methyl}-2-methyl-3-isothiazolidinecarboxamide 1,1-dioxide;
N-[(2-chlorophenyl)methyl]-2-(2-methylpropyl)-3-isothiazolidinecarboxamide 1,1-dioxide;
N-[(2-chloro-6-methylphenyl)methyl]-2-methyl-3-isothiazolidinecarboxamide 1,1-dioxide;
N-[(2,4-dichlorophenypmethyl]-2-methyl-3-isothiazolidinecarboxamide 1,1-dioxide;
2-ethyl-N-[(2,3,4-trifluorophenypmethyl]-3-isothiazolidinecarboxamide 1,1-dioxide;
N-[(2-chloro-4-fluorophenyl)methyl]-2-ethyl-3-isothiazolidinecarboxamide 1,1-dioxide;
2-methyl-N-[(2,3,4-trifluorophenyl)methyl]-3-isothiazolidinecarboxamide 1,1-dioxide;
N-[(3-chloro-2-methylphenyl)methyl]-2-methyl-3-isothiazolidinecarboxamide 1,1-dioxide;
N-[(3-chloro-2-methylphenyl)methyl]-2-(1-methylethyl)-3-isothiazolidinecarboxamide 1,1-dioxide;
N-[(3-chloro-2-methylphenyl)methyl]-2-(2-methylpropyl)-3-isothiazolidinecarboxamide 1,1-dioxide;
N-[(3-ehloro-2-methylphenyl)methyl]-2-ethyl-3-isothiazolidinecarboxamide 1,1-dioxide;
2-ethyl-N-[4-fluoro-2-(trifluoromethyl)phenyl]methyl)-3-isothiazolidinecarboxamide 1,1-dioxide;
N-[(2-chloro-4-fluorophenyl)methyl]-2-(1-methylethyl)-3-isothiazolidinecarboxamide 1,1-dioxide;
2-(1-methylethyl)-N-[(2,3,4-trifluorophenyl)methyl]-3-isothiazolidinecarboxamide 1,1-dioxide;
N-[(2,4-dichlorophenyl)methyl]-2-ethyl-3-isotlaiazolidinecarboxamide 1,1-dioxide;
2-(2-methylpropyl)-N-[(2,3,4-trifluorophenyl)methyl]-3-isothiazolidinecarboxamide 1,1-dioxide;
N-[(2-chloro-6-methylphenyl)methyl]-2-(1-methylethyl)-3-isothiazolidinecarboxamide 1,1-dioxide;
N-[(2,4-dichlorophenyl)methyl]-2-(1-methylethyl)-3-isothiazolidinecarboxamide 1,1-dioxide;
N-[(2-chloro-6-methylphenyl)methyl]-2-ethyl-3-isothiazolidinecarboxamide 1,1-dioxide;
N-{[2-chloro-3-(trifluoromethyl)phenyl]methyl}-2-methyl-3-isothiazolidinecarboxamide 1,1-dioxide;
N-[(2-chloro-3,4-difluorophenyl)methyl]-2-methyl-3-isothiazolidinecarboxamide 1,1-dioxide;
N-[(2-chloro-3,4-difluorophenyl)methyl]-2-ethyl-3-isothiazolidinecarboxamide 1,1-dioxide;
N-{[2-chloro-3-(trifluoromethyl)phenyl]methyl}-2-ethyl-3-isothiazolidinecarboxamide 1,1-dioxide;
2-ethyl-N-{[4-fluoro-2-(trifluoromethyl)phenyl]methyl}tetrahydro-2H-1,2-thiazine-3-carboxamide 1,1-dioxide;
N-[(2-chloro-4-fluorophenyl)methyl]-2-ethyltetrahydro-2H-1,2-thiazine-3-carboxamide 1,1-dioxide;
N-[(2,4-dichlorophenyl)methyl]-2-methyltetrahydro-2H-1,2-thiazine-3-carboxamide 1,1-dioxide;
N-[(2,4-dichlorophenyl)methyl]-2-ethyltetrahydro-2H-1,2-thiazine-3-carboxamide 1,1-dioxide;
N-[(2-chloro-4-fluorophenyl)methyl]-2-methyltetrahydro-2H-1,2-thiazine-3-carboxamide 1,1-dioxide;
N-[(3-chloro-2-methylphenyl)methyl]-2-ethyltetrahydro-2H-1,2-thiazine-3-carboxamide 1,1-dioxide;
N-[(2-chlorophenyl)methyl]-2-ethyltetrahydro-2H-1,2-thiazine-3-carboxamide 1,1-dioxide;
N-[(2,4-dichlorophenyl)methyl]-2-(1-methylethyl)tetrahydro-2H-1,2-thiazine-3-carboxamide 1,1-dioxide;
N-[(2-chloro-6-methylphenyl)methyl]-2-(1-methylethyl)tetrahydro-2H-1,2-thiazine-3-carboxamide 1,1-dioxide;
N-[(3-chloro-2-methylphenyl)methyl]-2-(1-methylethyl)tetrahydro-2H-1,2-thiazine-3-carboxamide 1,1-dioxide;
N-[(2-chloro-4-fluorophenyl)methyl]-2-(1-methylethyl)tetrahydro-2H-1,2-thiazine-3-carboxamide 1,1-dioxide;
N-[(2-chlorophenyl)methyl]-2-(1-methylethyl)tetrahydro-2H-1,2-thiazine-3-carboxamide 1,1-dioxide;
N-[(2-chloro-6-methylphenyl)methyl]-2-ethyltetrahydro-2H-1,2-thiazine-3-carboxamide 1,1-dioxide;
2-(1-methylethyl)-N-[(2,3,4-trifluorophenyl)methyl]tetrahydro-2H-1,2-thiazine-3-carboxamide 1,1-dioxide;
N-[4-fluoro-2-(trifluoromethyl)phenyl]methyl-2-(1-methylethyl)tetrahydro-2H-1,2-thiazine-3-carboxamide 1,1-dioxide;
N-[4-fluoro-2-(trifluoromethyephenyl)methyl]-2-methyltetrahydro-2H-1,2-thiazine-3-carboxamide 1,1-dioxide;
N-[(3-chloro-2-methylphenyl)methyl]-2-methyltetrahydro-2H-1,2-thiazine-3-carboxamide 1,1-dioxide;
N-[(2-chlorophenypmethyl]-2-methyltetrahydro-2H-1,2-thiazine-3-carboxamide 1,1-dioxide;
2-ethyl-N-[(2,3,4-trifluorophenyl)methyl]tetrahydro-2H-1,2-thiazine-3-carboxamide 1,1-dioxide;
N-[(2-chloro-6-methylphenyl)methyl]-2-methyltetrahy dro-2H-1,2-thiazine-3-carboxamide 1,1-dioxide; or
2-methyl-N-[(2,3,4-trifluorophenyl)methyl]tetrahydro-2H-1,2-thiazine-3-carboxamide 1,1-dioxide.

32. A pharmaceutical composition which comprises the compound or salt as defined in claim 19, and a pharmaceutically acceptable carrier or excipient.

33. A method of treating a human suffering from pain, rheumatoid arthritis, or osteoarthritis, which method comprises administering to said human an effective amount of the compound or salt as defined in claim 19.

34. A method of treating a human suffering from Alzheimer's disease or mild cognitive impairment associated with aging, which method comprises administering to said human an effective amount of the compound or salt as defined in claim 19.

35. A method of treating a human suffering from senile dementia, dementia with Lewy bodies, Pick's disease, Huntingdon's chorea, Parkinson's disease Creutzfeldt-Jakob disease, Amyotrophic Lateral Sclerosis, motor neuron disease, or multi-infarct dementia, which method comprises administering to said human an effective amount of the compound or salt as defined in claim 19.
